(19) United States
(12) Patent Application Publication
(10) Pub. No.: US 2024/0390373 A1
(43) Pub. Date: Nov. 28, 2024

(54) RAF KINASE INHIBITORS FOR TREATING TUMORS HARBORING GENE FUSIONS

(71) Applicant: Day One Biopharmaceuticals, Inc., Bribane, CA (US)

(72) Inventors: Samuel C. BLACKMAN, Eastsound, WA (US); Eleni VENETSANAKOS, San Bruno, CA (US); Michael C. COX, San Francisco, CA (US)

(21) Appl. No.: 18/696,942

(22) PCT Filed: Sep. 30, 2022
(86) PCT No.: PCT/US2022/045454

§ 371 Date: Mar. 28, 2024

Related U.S. Application Data
(60) Provisional application No. 63/251,445, filed on Oct. 1, 2021.

(51) Int. Cl. A61K 31/506; A61K 31/135; A61P 35/00; C12Q 1/6886

(52) U.S. Cl. CPC A61K 31/506; A61K 31/135; A61P 35/00; C12Q 1/6886; C12Q 2600/106; C12Q 2600/156

(57) ABSTRACT

Described herein are methods and compositions for treating tumors harboring gene fusions. In some embodiments, the present disclosure provides a method of treating tumors harboring gene fusions comprising administering to a subject in need thereof a Raf kinase inhibitor or a pharmaceutically acceptable salt thereof. In some embodiments, the Raf kinase inhibitor or a pharmaceutically acceptable salt thereof is (R)-2-(1-(6-amino-5-chloropyrimidine-4-carboxamido)ethyl)-N-(5-chloro-4-(trifluoromethyl)pyridin-2-yl) thiazole-5-carboxamide. In some embodiments, the gene fusion is a CRAF gene fusion, a BRAF gene fusion, or both.

### CROSS-REFERENCE

This application claims the benefit of U.S. Provisional Application Ser. No. 63/251,445 filed on Oct. 1, 2021, the entirety of which is hereby incorporated by reference herein.

### BACKGROUND

In 2012, there were an estimated 14.1 million cancer cases around the world. This number is expected to increase to 24 million by 2035. In 2014, there were an estimated 1,665,540 new cancer cases diagnosed and 585,720 cancer deaths in the U.S. Cancer remains the second most common cause of death in the U.S., accounting for nearly 1 of every 4 deaths. Although medical advances have improved cancer survival rates, there is a continuing need for new and more effective treatment.

### SUMMARY

In certain aspects, the present disclosure provides a method of treating cancer, the method comprising administering a Raf kinase inhibitor to a subject in need thereof, wherein the subject is identified having a gene fusion selected from AGK: BRAF and SNX8: BRAF. In some embodiments, the subject is identified having AGK: BRAF gene fusion. In some embodiments, the subject is identified having SNX8: BRAF gene fusion. In some embodiments, the Raf kinase inhibitor is (R)-2-(1-(6-amino-5-chloropyrimidine-4-carboxamido)ethyl)-N-(5-chloro-4-(trifluoromethyl) pyridin-2-yl) thiazole-5-carboxamide (Compound A).

In certain aspects, the present disclosure provides a method of treating cancer comprising administering a Raf kinase inhibitor to a subject in need thereof, wherein the subject is identified having one or more of the following gene fusions: SNX8: BRAF, STARD3NL: BRAF, BCAS1: BRAF, KHDRBS2: BRAF, CCDC6: BRAF, FAM131B: BRAF, SRGAP: BRAF, CLCN6: BRAF, GNAI1: BRAF, MRKN1: BRAF, GIT2: BRAF, GTF2I: BRAF, FXR1: BRAF, RNF130: BRAF, MACF1: BRAF, TMEM106B: BRAF, PPC1CC: BRAF, CUX1: BRAF, CCD6: BRAF, PPP1CC: BRAF, SEPT7: BRAF, PDE10A: BRAF, EPB41L2: BRAF, OSBP: BRAF, DAAMI: BRAF, TEX41: BRAF, FOXN3: BRAF, TRIPP1: BRAF, TOMIL2: BRAF, 5 BRAF fusions, QK1: RAF1, FYCO: RAF1, ATG7: RAF1, NFIA: RAF1, TMF1: RAF1, GOLGA3: RAF1, SOX6: RAF1, BMPRIA: RAF1, PDZRN3: RAF1, SLMAP: RAF1, MAP4: RAF1, BCL6: RAF1, SEPT17: BRAF, ZNF767: BRAF, CCDC91: BRAF, DYNC1/2: BRAF, ZKSCAN1: BRAF, MZT1: BRAF, RAD18: BRAF, CUX1: RAF1, CUL1: BRAF, SLC12A7: BRAF, TRIM24: BRAF, AGAP3: BRAF, AKAP9: BRAF, TAXIBP1: BRAF, CDC27: BRAF, FKBP15: BRAF, SKAP2: BRAF, TARDBP: BRAF, SEPT3: BRAF, ARMC10: BRAF, PAPSS1: BRAF, FCHSD1: BRAF, ERC1: BRAF, CDK5RAP2: BRAF, TMEM178B: BRAF, BAIAP2L1: BRAF, CEP89: BRAF, CNTNAP2: BRAF, EML4: BRAF, KCTD7: BRAF, LSM14A: BRAF, NFIC: BRAF, NUDCD3: BRAF, PHTF2: BRAF, PLIN3: BRAF, RP2: BRAF, SOX5: BRAF, SOX6: BRAF, TLK2: BRAF, ZKSCAN5: BRAF, KLC1: RAF1, DAAMI: RAF1, ZNF444: RAF1, LRCH3: RAF1, GOLGA4: RAF1, CTDSPL: RAF1, PRKAR2A: RAF1, CTNNA1: RAF1, MKRN1: RAF1, DYNC1H1: RAF1, GPHN: RAF1, KLHL7: BRAF, TANK: BRAF, RBMS3: BRAF, FAM114A2: BRAF, AGGF1: RAF1, EPS15: BRAF, NUP214: BRAF, BTF3L4: BRAF, GHR: BRAF, DOCK4: BRAF, ZC3HAV1: BRAF, MKRN1: BRAF, MYRIP: BRAF, SND1: BRAF, TNS3: BRAF, ATG7: BRAF, NUB1: BRAF, STRN3: BRAF, STK35: BRAF, ETFA: BRAF, SVOPL: BRAF, JHDMID: BRAF, PPFIBP2: BRAF, SCL45A3: BRAF, ESRP1: RAF1, AGTRAP: BRAF, SVIP: BRAF, NRF1: BRAF, RAF1: CCDC176, RAF1: TRAK1, ESYT2: BRAF, PCBP2: BRAF, or SALL2: BRAF. In some embodiments, the method comprises identifying the subject having the one or more gene fusions. In some embodiments, the method further comprises obtaining a cancer sample from the subject and subjecting the cancer sample to genomic testing prior to the administering of the Raf kinase inhibitor. In some embodiments, the subject is identified having a gene fusion that is SNX8: BRAF. In some embodiments, the RAF kinase inhibitor is selected from: vemurafenib, sorafenib, donafenib, dabrafenib, regorafenib, doramapimod, encorafenib, agerafenib, encorafenib, naporafenib, lifirafenib, belvarafenib, TAK632, CCT3833, RAF265, RAF709, trametinib, cobimetinib, LY3009120, LSN3074753, BGB659, CCT196969, CCT241161, INU152, PLX7904, PLX8394, PLX4720, BI882370, GDC0879, AZ 628, AZ 304, NVP-BHG712, SB590885, MLN2480, L-779450, ZM 336372, GW5074, CEP32496, B-RAF Inhibitor 1, MCP110, BAW2881, RO5126766, and (R)-2-(1-(6-amino-5-chloropyrimidine-4-carboxamido)ethyl)-N-(5-chloro-4-(trifluoromethyl) pyridin-2-yl) thiazole-5-carboxamide (Compound A). In some embodiments, the RAF kinase inhibitor is Compound A.

In certain aspects, the present disclosure provides a method of treating cancer, the method comprising administering a Raf kinase inhibitor to a subject in need thereof, wherein the subject has a CRAF gene fusion, wherein the Raf kinase inhibitor is (R)-2-(1-(6-amino-5-chloropyrimidine-4-carboxamido)ethyl)-N-(5-chloro-4-(trifluoromethyl) pyridin-2-yl) thiazole-5-carboxamide (Compound A) or a pharmaceutically acceptable salt thereof. In some embodiments, the CRAF gene fusion is SRGAP3: RAF1, QK1: RAF1, FYCO: RAF1, ATG7: RAF1, NFIA: RAF1, TMF1: RAF1, GOLGA3: RAF1, SOX6: RAF1, BMPRIA: RAF1, PDZRN3: RAF1, SLMAP: RAF1, MAP4: RAF1, BCL6: RAF1, CUX1: RAF1, KLC1: RAF1, DAAMI: RAF1, ZNF444: RAF1, LRCH3: RAF1, GOLGA4: RAF1, CTDSPL: RAF1, PRKAR2A: RAF1, CTNNA1: RAF1, MKRN1: RAF1, DYNCIH1: RAF1, AGGF1: RAF1, ESRP1: RAF1, GPHN: RAF1, RAF1-CCDC176, or RAF1-TRAK1. In some embodiments, the method comprises identifying the subject having the CRAF gene fusion. In some embodiments, the method further comprises obtaining a cancer sample from the subject and subjecting the cancer sample to genomic testing prior to the administering of Compound A. In some embodiments, the method further comprises administering a Raf kinase inhibitor to a subject in need thereof, wherein the subject has one or more of the following gene fusions: KIAA1549: BRAF, AGK: BRAF, STARD3NL: BRAF, BCAS1: BRAF, KHDRBS2: BRAF, CCDC6: BRAF, FAM131B: BRAF, SRGAP: BRAF, CLCN6: BRAF, GNAI1: BRAF, MRKN1: BRAF, GIT2: BRAF, GTF21: BRAF, FXR1: BRAF, RNF130: BRAF, MACF1: BRAF, TMEM106B: BRAF, PPC1CC: BRAF, CUX1: BRAF, CCD6: BRAF, PPP1CC: BRAF, SEPT7: BRAF, PDE10A: BRAF, EPB41L2: BRAF, OSBP: BRAF, DAAMI: BRAF, TEX41: BRAF, FOXN3: BRAF, TRIPP1: BRAF, TOMIL2: BRAF, TMEM106B: BRAF, SRGAP3: RAF1, QK1: RAF1, FYCO: RAF1, ATG7: RAF1, NFIA-RAF1, TMF1: RAF1, GOLGA3: RAF1, SOX6: RAF1, BMPRIA: RAF1, PDZRN3: RAF1, SLMAP: RAF1, MAP4: RAF1, BCL6-RAF1, SEPT17: BRAF, ZNF767: BRAF, CCDC91: BRAF, DYNC1/2: BRAF, ZKSCAN1: BRAF, GTF2I: BRAF, MZT1: BRAF, RAD18: BRAF, CUX1: BRAF, CUX1-RAF1, CUL1: BRAF, SLC12A7: BRAF, TRIM24: BRAF, AGAP3: BRAF, AKAP9: BRAF, TAXIBP1: BRAF, CDC27: BRAF, FKBP15: BRAF, SKAP2: BRAF, TARDBP: BRAF, SEPT3: BRAF, ARMC10: BRAF, PAPSS1: BRAF, FCHSD1: BRAF, ERC1: BRAF, CDK5RAP2: BRAF, TMEM178B: BRAF, BAIAP2L1: BRAF, CEP89: BRAF, CNTNAP2: BRAF, EML4: BRAF, KCTD7: BRAF, LSM14A: BRAF, NFIC: BRAF, NUDCD3: BRAF, PHTF2: BRAF, PLIN3: BRAF, RP2: BRAF, SOX5: BRAF, SOX6: BRAF, TLK2: BRAF, ZKSCAN5: BRAF, KLC1-RAF1, DAAMI-RAF1, ZNF444-RAF1, LRCH3-RAF1, GOLGA4-RAF1, CTDSPL-RAF1, PRKAR2A-RAF1, CTNNA1-RAF1, MKRN1-RAF1, DYNCIH1-RAF1, GPHN-RAF1, KLHL7: BRAF, TANK: BRAF, RBMS3: BRAF, FAM114A2: BRAF, AGGF1-RAF1, EPS15: BRAF, NUP214: BRAF, BTF3L4: BRAF, GHR: BRAF, DOCK4: BRAF, ZC3HAV1: BRAF, MKRN1: BRAF, MYRIP: BRAF, SND1: BRAF, TNS3: BRAF, ATG7: BRAF, NUB1: BRAF, STRN3: BRAF, STK35: BRAF, ETFA: BRAF, SVOPL: BRAF, JHDMID: BRAF, PPFIBP2: BRAF, SCL45A3: BRAF, ESRP1-RAF1, AGTRAP: BRAF, SVIP: BRAF, NRF1: BRAF, RAF1-CCDC176, RAF1-TRAK1, ESYT2: BRAF, PCBP2: BRAF, and SALL2: BRAF, wherein the Raf kinase inhibitor is (R)-2-(1-(6-amino-5-chloropyrimidine-4-carboxamido)ethyl)-N-(5-chloro-4-(trifluoromethyl) pyridin-2-yl) thiazole-5-carboxamide (Compound A) or a pharmaceutically acceptable salt thereof, and wherein the subject is at least 18 years of age.

In certain aspects, the present disclosure provides a method of treating solid tumors in an adult subject, the method comprising administering a Raf kinase inhibitor to a subject in need thereof, wherein the subject has a CRAF gene fusion, a BRAF gene fusion, or both, wherein the Raf kinase inhibitor is (R)-2-(1-(6-amino-5-chloropyrimidine-4-carboxamido)ethyl)-N-(5-chloro-4-(trifluoromethyl) pyridin-2-yl) thiazole-5-carboxamide (Compound A) or a pharmaceutically acceptable salt thereof, wherein the subject is at least 18 years of age. In some embodiments, the subject has a gene fusion that is KIAA1549: BRAF or SRGAP3: RAF1.

In certain aspects, the present disclosure provides a method of treating cancer, the method comprising administering a Raf kinase inhibitor to a subject in need thereof, wherein the subject has one or more of the following gene fusions: AGK: BRAF, STARD3NL: BRAF, BCAS1: BRAF, KHDRBS2: BRAF, CCDC6: BRAF, FAM131B: BRAF, SRGAP: BRAF, CLCN6: BRAF, GNAI1: BRAF, MRKN1: BRAF, GIT2: BRAF, GTF2I: BRAF, FXR1: BRAF, RNF130: BRAF, MACF1: BRAF, TMEM106B: BRAF, PPC1CC: BRAF, CUX1: BRAF, CCD6: BRAF, PPP1CC: BRAF, SEPT7: BRAF, PDE10A: BRAF, EPB41L2: BRAF, OSBP: BRAF, DAAMI: BRAF, TEX41: BRAF, FOXN3: BRAF, TRIPP1: BRAF, TOMIL2: BRAF, TMEM106B: BRAF, QK1: RAF1, FYCO: RAF1, ATG7: RAF1, NFIA-RAF1, TMF1: RAF1, GOLGA3: RAF1, SOX6: RAF1, BMPRIA: RAF1, PDZRN3: RAF1, SLMAP: RAF1, MAP4: RAF1, BCL6-RAF1, SEPT17: BRAF, ZNF767: BRAF, CCDC91: BRAF, DYNC1/2: BRAF, ZKSCAN1: BRAF, GTF2I: BRAF, MZT1: BRAF, RAD18: BRAF, CUX1: BRAF, CUX1-RAF1, CUL1: BRAF, SLC12A7: BRAF, TRIM24: BRAF, AGAP3: BRAF, AKAP9: BRAF, TAXIBP1: BRAF, CDC27: BRAF, FKBP15: BRAF, SKAP2: BRAF, TARDBP: BRAF, SEPT3: BRAF, ARMC10: BRAF, PAPSS1: BRAF, FCHSD1: BRAF, ERC1: BRAF, CDK5RAP2: BRAF, TMEM178B: BRAF, BAIAP2L1: BRAF, CEP89: BRAF, CNTNAP2: BRAF, EML4: BRAF, KCTD7: BRAF, LSM14A: BRAF, NFIC: BRAF, NUDCD3: BRAF, PHTF2: BRAF, PLIN3: BRAF, RP2: BRAF, SOX5: BRAF, SOX6: BRAF, TLK2: BRAF, ZKSCAN5: BRAF, KLC1-RAF1, DAAMI-RAF1, ZNF444-RAF1, LRCH3-RAF1, GOLGA4-RAF1, CTDSPL-RAF1, PRKAR2A-RAF1, CTNNA1-RAF1, MKRN1-RAF1, DYNC1H1-RAF1, GPHN-RAF1, KLHL7: BRAF, TANK: BRAF, RBMS3: BRAF, FAM114A2: BRAF, AGGF1-RAF1, EPS15: BRAF, NUP214: BRAF, BTF3L4: BRAF, GHR: BRAF, DOCK4: BRAF, ZC3HAV1: BRAF, MKRN1: BRAF, MYRIP: BRAF, SND1: BRAF, TNS3: BRAF, ATG7: BRAF, NUB1: BRAF, STRN3: BRAF, STK35: BRAF, ETFA: BRAF, SVOPL: BRAF, JHDMID: BRAF, PPFIBP2: BRAF, SCL45A3: BRAF, ESRP1-RAF1, AGTRAP: BRAF, SVIP: BRAF, NRF1: BRAF, RAF1-CCDC176, RAF1-TRAK1, ESYT2: BRAF, PCBP2: BRAF, or SALL2: BRAF, and wherein the Raf kinase inhibitor is (R)-2-(1-(6-amino-5-chloropyrimidine-4-carboxamido)ethyl)-N-(5-chloro-4-(trifluoromethyl) pyridin-2-yl) thiazole-5-carboxamide (Compound A) or a pharmaceutically acceptable salt thereof. In some embodiments, the gene fusion is a BRAF fusion. In some embodiments, the BRAF fusion is selected from: AGK: BRAF, STARD3NL: BRAF, BCAS1: BRAF, KHDRBS2: BRAF, CCDC6: BRAF, FAM131B: BRAF, SRGAP: BRAF, CLCN6: BRAF, GNAI1: BRAF, MRKN1: BRAF, GIT2: BRAF, GTF2I: BRAF, FXR1: BRAF, RNF130: BRAF, MACF1: BRAF, TMEM106B: BRAF, PPC1CC: BRAF, CUX1: BRAF, CCD6: BRAF, PPP1CC: BRAF, SEPT7: BRAF, PDE10A: BRAF, EPB41L2: BRAF, OSBP: BRAF, DAAMI: BRAF, TEX41: BRAF, FOXN3: BRAF, TRIPP1: BRAF, TOMIL2: BRAF, 5 BRAF fusions, TMEM106B: BRAF, SEPT17: BRAF, ZNF767: BRAF, CCDC91: BRAF, DYNC1/2: BRAF, ZKSCAN1: BRAF, GTF2I: BRAF, MZT1: BRAF, RAD18: BRAF, CUX1: BRAF, CUL1: BRAF, SLC12A7: BRAF, TRIM24: BRAF, AGAP3: BRAF, AKAP9: BRAF, TAXIBP1: BRAF, CDC27: BRAF, FKBP15: BRAF, SKAP2: BRAF, TARDBP: BRAF, SEPT3: BRAF, ARMC10: BRAF, PAPSS1: BRAF, FCHSD1: BRAF, ERC1: BRAF, CDK5RAP2: BRAF, TMEM178B: BRAF, BAIAP2L1: BRAF, CEP89: BRAF, CNTNAP2: BRAF, EML4: BRAF, KCTD7: BRAF, LSM14A: BRAF, NFIC: BRAF, NUDCD3: BRAF, PHTF2: BRAF, PLIN3: BRAF, RP2: BRAF, SOX5: BRAF, SOX6: BRAF, TLK2: BRAF, ZKSCAN5: BRAF, KLHL7: BRAF, TANK: BRAF, RBMS3: BRAF, FAM114A2: BRAF, EPS15: BRAF, NUP214: BRAF, BTF3L4: BRAF, GHR: BRAF, DOCK4: BRAF, ZC3HAV1: BRAF, MKRN1: BRAF, MYRIP: BRAF, SND1: BRAF, TNS3: BRAF, ATG7: BRAF, NUB1: BRAF, STRN3: BRAF, STK35: BRAF, ETFA: BRAF, SVOPL: BRAF, JHDMID: BRAF, PPFIBP2: BRAF, SCL45A3: BRAF, AGTRAP: BRAF, SVIP: BRAF, NRF1: BRAF, ESYT2: BRAF, PCBP2: BRAF, and SALL2: BRAF. In some embodiments, the gene fusion is a RAF1 fusion. In some embodiments, the RAF1 fusion is selected from: SRGAP3: RAF1, QK1: RAF1, FYCO: RAF1, ATG7: RAF1, NFIA: RAF1, TMF1: RAF1, GOLGA3: RAF1, SOX6: RAF1, BMPRIA: RAF1, PDZRN3: RAF1, SLMAP: RAF1, MAP4: RAF1, BCL6: RAF1, CUX1: RAF1, KLC1: RAF1, DAAMI: RAF1, ZNF444: RAF1, LRCH3: RAF1, GOLGA4: RAF1, CTDSPL: RAF1, PRKAR2A: RAF1, CTNNA1: RAF1, MKRN1: RAF1, DYNCIH1: RAF1, AGGF1: RAF1, ESRP1: RAF1, GPHN: RAF1, RAF1-CCDC176, and RAF1-TRAK1.

In certain aspects, the present disclosure provides a method of treating a solid tumor in an adult subject, the method comprising administering a Raf kinase inhibitor to a subject in need thereof wherein the subject has a wild-type CRAF gene fusion, a wild-type BRAF gene fusion, or both; and wherein the Raf kinase inhibitor is (R)-2-(1-(6-amino-5-chloropyrimidine-4-carboxamido)ethyl)-N-(5-chloro-4-(trifluoromethyl) pyridin-2-yl) thiazole-5-carboxamide (Compound A) or a pharmaceutically acceptable salt thereof.

In certain aspects the cancer described herein is a solid tumor. In some embodiments, the solid tumor is an extracranial tumor. In some embodiments, the solid tumor is a soft tissue sarcoma. In some embodiments, the solid tumor is a bone sarcoma. In some embodiments, the bone sarcoma is selected from osteosarcoma, chonrosarcoma, spindle cell sarcoma, hemangioendothelioma, angiosarcoma, fibrosarcoma, chordoma, adamantinoma, liposarcoma, leiomyosarcoma, malignant peripheral nerve sheath tumor, rhabdomyosarcoma, synovial sarcoma, and malignant solitary fibrous tumor. In some embodiments, the bone sarcoma is a spindle cell sarcoma. In some embodiments, the cancer is an advanced solid tumor. In some embodiments, the cancer is a recurrent cancer. In some embodiments, the subject has received at least one prior therapy before the administration of the (R)-2-(1-(6-amino-5-chloropyrimidine-4-carboxamido)ethyl)-N-(5-chloro-4-(trifluoromethyl) pyridin-2-yl) thiazole-5-carboxamide (Compound A) or a pharmaceutically acceptable salt thereof. In some embodiments, the prior therapy is chemotherapy therapy, hormone therapy, immunotherapy, or radiation therapy. In some embodiments, the method further comprises obtaining a cancer sample from the subject and subjecting the cancer sample to genomic testing prior to the administering of Compound A. In some embodiments, the genomic profiling is whole exome sequencing. In some embodiments, the method further comprises the administration of a dermatological agent. In some embodiments, the dermatological agent is diphenhydramine. In some embodiments, the administrating occurs in a plurality of treatment stages which alternate between an administration stage and a non-administration stage. In some embodiments, the administrating occurs in a plurality of treatment stages which alternate between an administration stage and a non-administration stage. In some embodiments, the administration stage has a duration of 28 days. In some embodiments, the administration stage comprises the administration of Compound A or a pharmaceutically acceptable salt thereof once weekly. In some embodiments, Compound A or a pharmaceutically acceptable salt thereof is administered at 420 mg/m². In some embodiments, the administering of Compound A or a pharmaceutically acceptable salt thereof comprises an initial dose of the Compound A or a pharmaceutically acceptable salt thereof equivalent to about 280 mg/m² to about 600 mg/m² of Compound A per week. In some embodiments, Compound A or a pharmaceutically acceptable salt thereof is administered in an amount of up to about 600 mg per dose. In some embodiments, Compound A or a pharmaceutically acceptable salt thereof is administered in an amount of up to about 200 mg per dose. In some embodiments, Compound A or a pharmaceutically acceptable salt thereof is administered on days 1, 3, 5, 8, 10, 12, 15, 17, 19, 22, 24, and 26 of a 28-day cycle.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the inventive concepts are set forth with particularity in the appended claims. A better understanding of the features and advantages of the present inventive concepts will be obtained by reference to the following detailed description that sets forth illustrative embodiments, in which the principles of the inventive concepts are utilized, and the accompanying drawings of which:

FIG. 1 illustrates anti-tumor activity of Compound A in AGK-BRAF fusion HuPrime® Melanoma Xenograft model ME11971.

### DETAILED DESCRIPTION

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the invention.

### Definitions

Unless defined otherwise, all technical and scientific terms used herein have the same meaning as is commonly understood by one of skill in the art to which this disclosure belongs. All patents and publications referred to herein are incorporated by reference.

As used in the specification and claims, the singular form “a”, “an” and “the” includes plural references unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure. Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below. As used herein, the terms “treatment,” “treat,” and “treating” are meant to include the full spectrum of intervention for the cancer from which the subject is suffering, such as administration of the combination to alleviate, slow, stop, or reverse one or more symptoms of the cancer and to delay the progression of the cancer even if the cancer is not actually eliminated. Treatment can include, for example, a decrease in the severity of a symptom, the number of symptoms, or frequency of relapse, e.g., the inhibition of tumor growth, the arrest of tumor growth, or the regression of already existing tumors.

As used herein, the term “Raf kinase” refers to any one of a family of serine/threonine-protein kinases. The family consists of three isoform members (B-Raf, C-Raf (Raf-1), and A-Raf). Raf protein kinases are involved in the MAPK signaling pathway consisting of a kinase cascade that relays extracellular signals to the nucleus to regulate gene expression and key cellular functions. Unless otherwise indicated by context, the term “Raf kinase” is meant to refer to any Raf kinase protein from any species, including, without limitation. In one aspect, the Raf kinase is a human Raf kinase.

The term “Raf inhibitor,” “Raf kinase inhibitor,” or “inhibitor of Raf” is used to signify a compound which is capable of interacting with one or more isoform members (B-Raf, C-Raf (Raf-1), and/or A-Raf) of the serine/threonine-protein kinase, Raf including mutant forms. Some examples of Raf mutant forms include, but are not limited to, B-Raf V600E, B-Raf V600D, B-Raf V600K, B-Raf V600E+T5291, and/or B-Raf V600E+G468A.

In some embodiments, the Raf kinase is at least about 50% inhibited, at least about 75% inhibited, at least about 90% inhibited, at least about 95% inhibited, at least about 98% inhibited, or at least about 99% inhibited. In some embodiments, the concentration of Raf kinase inhibitor required to reduce Raf kinase activity by 50% is less than about 1 μM, less than about 500 nM, less than about 100 nM, less than about 50 nM, less than about 25 nM, less than about 10 nM, less than about 5 nM, or less than about 1 nM.

In some embodiments, such inhibition is selective for one or more Raf isoforms, i.e., the Raf inhibitor is selective for one or more of B-Raf (wild type), mutant B-Raf, A-Raf, and C-Raf kinase. In some embodiments, the Raf inhibitor is selective for B-Raf (wild type), B-Raf V600E, A-Raf and C-Raf. In some embodiments, the Raf inhibitor is selective for B-Raf (wild type), B-Raf V600E, A-Raf, and C-Raf. In some embodiments, the Raf inhibitor is selective for B-Raf (wild type), B-Raf V600D, A-Raf, and C-Raf. In some embodiments, the Raf inhibitor is selective for B-Raf (wild type), B-Raf V600K, and C-Raf. In some embodiments, the Raf inhibitor is selective for more than B-Raf V600. In some embodiments, the Raf inhibitor is selective for more than B-Raf V600E.

In some embodiments, the Raf inhibitor is selective for B-Raf and C-Raf kinases. In some embodiments, the Raf inhibitor is selective for B-Raf (wild type), B-Raf V600E, and C-Raf. In some embodiments, the Raf inhibitor is selective for B-Raf (wild type), B-Raf V600D, and C-Raf. In some embodiments, the Raf inhibitor is selective for B-Raf (wild type), B-Raf V600K, and C-Raf. In some embodiments, the Raf inhibitor is selective for mutant B-Raf. In some embodiments, the Raf inhibitor is selective for mutant B-Raf V600E. In some embodiments, the Raf inhibitor is selective for mutant B-Raf V600D. In some embodiments, the Raf inhibitor is selective for mutant B-Raf V600K.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salt” or “pharmaceutically acceptable salt” refers to salts derived from a variety of organic and inorganic counter ions well known in the art. Pharmaceutically acceptable acid addition salts can be formed with inorganic acids and organic acids. Pharmaceutically acceptable base addition salts can be formed with inorganic and organic bases. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

Unless otherwise stated, structures depicted herein are meant to include compounds which differ only in the presence of one or more isotopically enriched atoms. For example, compounds having the present structure except for the replacement of a hydrogen atom by a deuterium or tritium, or the replacement of a carbon atom by a 13C- or 14C-enriched carbon are within the scope of the disclosure.

Certain compounds described herein may exist in tautomeric forms, and all such tautomeric forms of the compounds being within the scope of the disclosure. Unless otherwise stated, structures depicted herein are also meant to include all stereochemical forms of the structure. i.e., the R and S configurations for each asymmetric center. Therefore, single stereochemical isomers as well as enantiomeric and diastereomeric mixtures of the present compounds are within the scope of the disclosure.

The terms “subject,” “individual,” and “patient” may be used interchangeably and refer to humans as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, and female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a compound or salt described herein.

The terms “administer”, “administered”, “administers,” and “administering” are defined as providing a composition to a subject via a route known in the art, including, but not limited to, intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used. The terms “administer”, “administered”, “administers” and “administering” a compound should be understood to mean providing a compound of the disclosure or a prodrug of a compound of the disclosure to the individual in need.

As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a compound or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treating can include, for example, reducing, delaying, or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition and can contemplate a range of results directed to that end, including, but not restricted to, prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

### Treatment of Cancers Harboring Gene Fusions

In one aspect, the present disclosure provides methods of treating a disease or condition (such as a cancer) comprising administering a Raf kinase inhibitor to a subject in need thereof, wherein the subject is identified having one or more gene fusions. BRAF and/or CRAF wild-type gene fusions can be predictive of response to Raf kinase inhibitors (such as Compound A) in certain diseases or conditions (including tumors, e.g., extracranial solid tumors). Exemplary BRAF and CRAF gene fusions of the present disclosure include, but are not limited to, SNX8: BRAF, STARD3NL: BRAF, BCAS1: BRAF, KHDRBS2: BRAF, CCDC6: BRAF, FAM131B: BRAF, SRGAP: BRAF, CLCN6: BRAF, GNAI1: BRAF, MRKN1: BRAF, GIT2: BRAF, GTF2I: BRAF, FXR1: BRAF, RNF130: BRAF, MACF1: BRAF, TMEM106B: BRAF, PPC1CC: BRAF, CUX1: BRAF, CCD6: BRAF, PPP1CC: BRAF, SEPT7: BRAF, PDE10A: BRAF, EPB41L2: BRAF, OSBP: BRAF, DAAMI: BRAF, TEX41: BRAF, FOXN3: BRAF, TRIPP1: BRAF, TOMIL2: BRAF, QK1: RAF1, FYCO: RAF1, ATG7: RAF1, NFIA: RAF1, TMF1: RAF1, GOLGA3: RAF1, SOX6: RAF1, BMPRIA: RAF1, PDZRN3: RAF1, SLMAP: RAF1, MAP4: RAF1, BCL6: RAF1, SEPT17: BRAF, ZNF767: BRAF, CCDC91: BRAF, DYNC1/2: BRAF, ZKSCAN1: BRAF, MZT1: BRAF, RAD18: BRAF, CUX1: RAF1, CUL1: BRAF, SLC12A7: BRAF, TRIM24: BRAF, AGAP3: BRAF, AKAP9: BRAF, TAXIBP1: BRAF, CDC27: BRAF, FKBP15: BRAF, SKAP2: BRAF, TARDBP: BRAF, SEPT3: BRAF, ARMC10: BRAF, PAPSS1: BRAF, FCHSD1: BRAF, ERC1: BRAF, CDK5RAP2: BRAF, TMEM178B: BRAF, BAIAP2L1: BRAF, CEP89: BRAF, CNTNAP2: BRAF, EML4: BRAF, KCTD7: BRAF, LSM14A: BRAF, NFIC: BRAF, NUDCD3: BRAF, PHTF2: BRAF, PLIN3: BRAF, RP2: BRAF, SOX5: BRAF, SOX6: BRAF, TLK2: BRAF, ZKSCAN5: BRAF, KLC1: RAF1, DAAMI: RAF1, ZNF444: RAF1, LRCH3: RAF1, GOLGA4: RAF1, CTDSPL: RAF1, PRKAR2A: RAF1, CTNNA1: RAF1, MKRN1: RAF1, DYNCIH1: RAF1, GPHN: RAF1, KLHL7: BRAF, TANK: BRAF, RBMS3: BRAF, FAM114A2: BRAF, AGGF1: RAF1, EPS15: BRAF, NUP214: BRAF, BTF3L4: BRAF, GHR: BRAF, DOCK4: BRAF, ZC3HAV1: BRAF, MKRN1: BRAF, MYRIP: BRAF, SND1: BRAF, TNS3: BRAF, ATG7: BRAF, NUB1: BRAF, STRN3: BRAF, STK35: BRAF, ETFA: BRAF, SVOPL: BRAF, JHDMID: BRAF, PPFIBP2: BRAF, SCL45A3: BRAF, ESRP1: RAF1, AGTRAP: BRAF, SVIP: BRAF, NRF1: BRAF, RAF1: CCDC176, RAF1: TRAK1, ESYT2: BRAF, PCBP2: BRAF, and SALL2: BRAF. In some embodiments, the method comprises identifying the subject having the one or more gene fusions. In some embodiments, the method comprises identifying the subject having the one or more BRAF or CRAF gene fusions. In some embodiments, the method comprises identifying the subject having the one or more wild-type BRAF or wild-type CRAF gene fusions. In some embodiments, the method comprises identifying a subject having a wild-type BRAF gene fusions. In some embodiments, the method comprises identifying a subject having a wild-type CRAF gene fusions. In some embodiments, the BRAF fusion is a wild-type BRAF fusion. In some embodiments, the CRAF gene fusion is a wild-type BRAF fusion. In some embodiments, the gene fusion disclosed herein is a canonical gene fusion. In some embodiments, the gene fusion disclosed herein is a non-canonical gene fusion. In some embodiments, the BRAF fusion disclosed herein is a non-canonical BRAF fusion (e.g., a gene fusion that is not KIAA1549 gene fusion). In some embodiments, the BRAF fusion disclosed herein is a non-canonical wild-type BRAF fusion. In some embodiments, the method comprises obtaining a cancer sample from the subject and subjecting the cancer sample to genomic testing prior to the administering of the Raf kinase inhibitor. In some embodiments, the subject is identified as having a gene fusion that is SNX8: BRAF. In some embodiments, the subject is identified as having a gene fusion that is RIN2: BRAF.

### A. Raf Kinase Inhibitors

In one aspect, a Raf kinase inhibitor described herein is a BRAF and/or a CRAF kinase inhibitor. In some embodiments, the Raf kinase inhibitor is selective for BRAF and CRAF. In some embodiments, the Raf kinase inhibitor is selective for BRAF or CRAF. In some embodiments, the method comprises identifying the subject having the one or more wild-type BRAF or wild-type CRAF gene fusions. In some embodiments, the BRAF fusion is a wild-type BRAF fusion. In some embodiments, the CRAF gene fusion is a wild-type BRAF fusion. In some embodiments, the Raf kinase inhibitor is selective for BRAF. In some embodiments, the Raf kinase inhibitor is selective for wild-type BRAF. In some embodiments, the Raf kinase inhibitor is selective for CRAF. In some embodiments, the Raf kinase inhibitor is selective for wild-type CRAF. In some embodiments, the Raf kinase inhibitor is selective toward gene fusions having one or more of a BRAF or a CRAF gene fusions. In some embodiments, the Raf kinase inhibitor is selective toward gene fusions having one or more of a BRAF gene fusion. In some embodiments, the Raf kinase inhibitor is selective toward gene fusions having one or more of a CRAF gene fusions. In some embodiments, a Raf kinase inhibitor described herein is a type I Raf inhibitor. In some embodiments, a Raf kinase inhibitor described herein is a type II Raf inhibitor. In some embodiments, a Raf kinase inhibitor described herein is a pan-Raf inhibitor.

Compounds capable of inhibiting the activity of a Raf kinase may be used in the methods of the instant disclosure. In some embodiments, the RAF kinase inhibitor is a compound as described in WO 2006/065703, WO 2010/064722, WO 2011/117381, WO 2011/090738, WO 2011/161216, WO 2011/097526, WO 2011/025927, WO 2011/023773, WO 2011/147764, WO 2011/079133, WO 2011/063159, WO 2009/006389, WO2006/06570, and US 2013/0252977, the entire contents of each of which are incorporated in their entireties.

In some embodiments, the RAF kinase inhibitor is selected from: vemurafenib, sorafenib, donafenib, dabrafenib, regorafenib, doramapimod, encorafenib, agerafenib, encorafenib, naporafenib, lifirafenib, belvarafenib, TAK632, CCT3833, RAF265, RAF709, trametinib, cobimetinib, LY3009120, LSN3074753, BGB659, CCT196969, CCT241161, INU152, PLX7904, PLX8394, PLX4720, BI882370, GDC0879, AZ 628, AZ 304, NVP-BHG712, SB590885, MLN2480, L-779450, ZM 336372, GW5074, CEP32496, B-RAF Inhibitor 1, MCP110, BAW2881, RO5126766, (R)-2-(1-(6-amino-5-chloropyrimidine-4-carboxamido)ethyl)-N-(5-chloro-4-(trifluoromethyl) pyridin-2-yl) thiazole-5-carboxamide (Compound A), and a pharmaceutically acceptable salt thereof. In some embodiments, the RAF kinase inhibitor is selected from: vemurafenib, sorafenib, donafenib, dabrafenib, regorafenib, doramapimod, encorafenib, agerafenib, encorafenib, naporafenib, lifirafenib, belvarafenib, TAK632, CCT3833, RAF265, RAF709, trametinib, cobimetinib, LY3009120, LSN3074753, BGB659, CCT196969, CCT241161, INU152, PLX7904, PLX8394, PLX4720, BI882370, GDC0879, AZ 628, AZ 304, NVP-BHG712, SB590885, MLN2480, L-779450, ZM 336372, GW5074, CEP32496, B-RAF Inhibitor 1, MCP110, BAW2881, RO5126766, and (R)-2-(1-(6-amino-5-chloropyrimidine-4-carboxamido)ethyl)-N-(5-chloro-4-(trifluoromethyl) pyridin-2-yl) thiazole-5-carboxamide (Compound A). Compound A is also known as tovorafenib, DAY101, TAK-580, and MLN2480.

In some embodiments, the RAF kinase inhibitor is selected from a compound of Table 1, or a pharmaceutically acceptable salt thereof:

TABLE 1
Raf Inhibitors

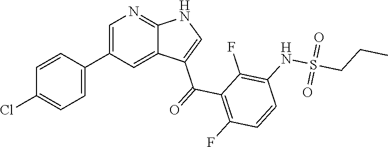 [SMILES: CCCS(=O)(=O)Nc1ccc(F)c(C(=O)c2c[nH]c3ncc(-c4ccc(Cl)cc4)cc23)c1F]

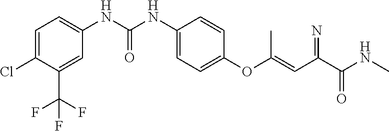

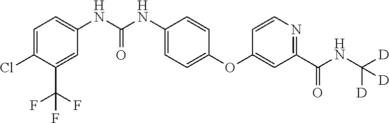 [SMILES: [2H]C([2H])([2H])NC(=O)c1cc(Oc2ccc(NC(=O)Nc3ccc(Cl)c(C(F)(F)F)c3)cc2)ccn1]

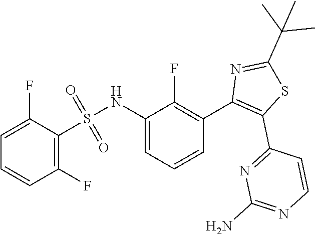 [SMILES: CC(C)(C)c1nc(-c2cccc(NS(=O)(=O)c3c(F)cccc3F)c2F)c(-c2ccnc(N)n2)s1]

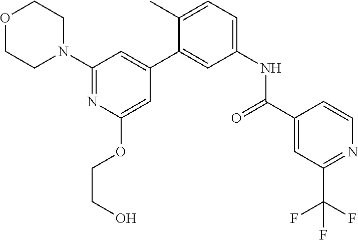 [SMILES: Cc1ccc(NC(=O)c2ccnc(C(F)(F)F)c2)cc1-c1cc(OCCO)nc(N2CCOCC2)c1]

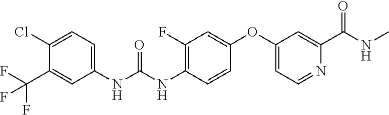 [SMILES: CNC(=O)c1cc(Oc2ccc(NC(=O)Nc3ccc(Cl)c(C(F)(F)F)c3)c(F)c2)ccn1]

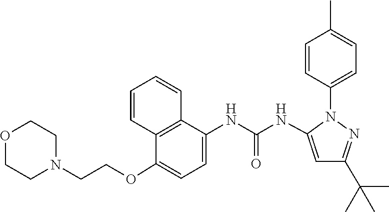 [SMILES: Cc1ccc(-n2nc(C(C)(C)C)cc2NC(=O)Nc2ccc(OCCN3CCOCC3)c3ccccc23)cc1]

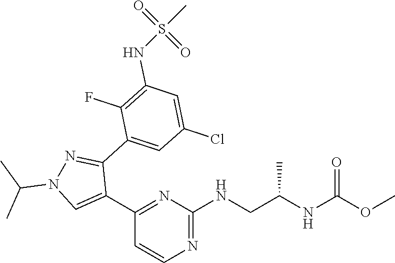 [SMILES: COC(=O)N[C@@H](C)CNc1nccc(-c2cn(C(C)C)nc2-c2cc(Cl)cc(NS(C)(=O)=O)c2F)n1]

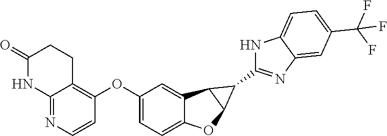 [SMILES: O=C1CCc2c(Oc3ccc4c(c3)[C@@H]3[C@H](O4)[C@H]3c3nc4cc(C(F)(F)F)ccc4[nH]3)ccnc2N1]

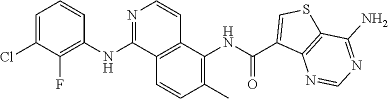 [SMILES: Cc1ccc2c(Nc3cccc(Cl)c3F)nccc2c1NC(=O)c1csc2c(N)ncnc12]

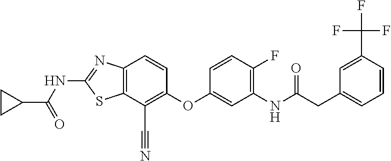 [SMILES: N#Cc1c(Oc2ccc(F)c(NC(=O)Cc3cccc(C(F)(F)F)c3)c2)ccc2nc(NC(=O)C3CC3)sc12]

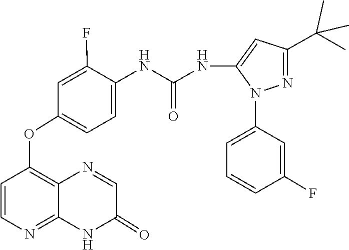 [SMILES: CC(C)(C)c1cc(NC(=O)Nc2ccc(Oc3ccnc4[nH]c(=O)cnc34)cc2F)n(-c2cccc(F)c2)n1]

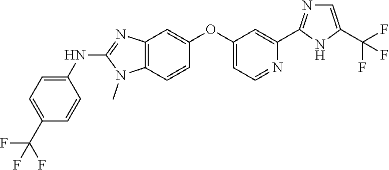 [SMILES: Cn1c(Nc2ccc(C(F)(F)F)cc2)nc2cc(Oc3ccnc(-c4ncc(C(F)(F)F)[nH]4)c3)ccc21]

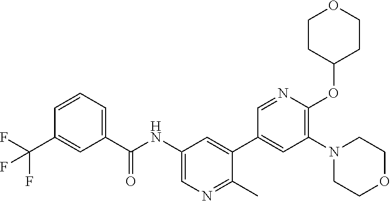 [SMILES: Cc1ncc(NC(=O)c2cccc(C(F)(F)F)c2)cc1-c1cnc(OC2CCOCC2)c(N2CCOCC2)c1]

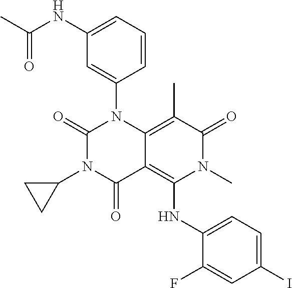 [SMILES: CC(=O)Nc1cccc(-n2c(=O)n(C3CC3)c(=O)c3c(Nc4ccc(I)cc4F)n(C)c(=O)c(C)c32)c1]

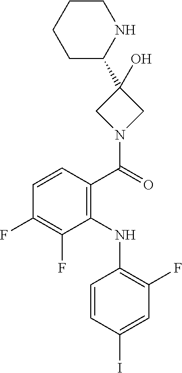 [SMILES: O=C(c1ccc(F)c(F)c1Nc1ccc(I)cc1F)N1CC(O)([C@@H]2CCCCN2)C1]

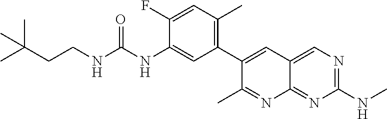 [SMILES: CNc1ncc2cc(-c3cc(NC(=O)NCCC(C)(C)C)c(F)cc3C)c(C)nc2n1]

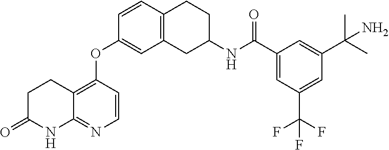 [SMILES: CC(C)(N)c1cc(C(=O)NC2CCc3ccc(Oc4ccnc5c4CCC(=O)N5)cc3C2)cc(C(F)(F)F)c1]

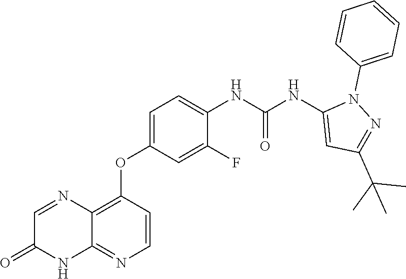 [SMILES: CC(C)(C)c1cc(NC(=O)Nc2ccc(Oc3ccnc4[nH]c(=O)cnc34)cc2F)n(-c2ccccc2)n1]

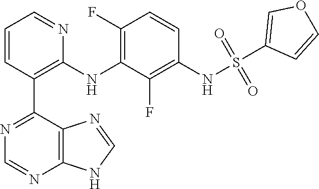 [SMILES: O=S(=O)(Nc1ccc(F)c(Nc2ncccc2-c2ncnc3[nH]cnc23)c1F)c1ccoc1]

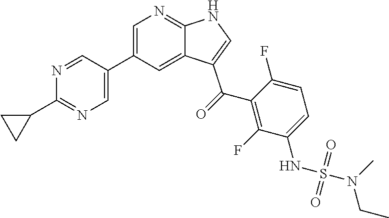 [SMILES: CCN(C)S(=O)(=O)Nc1ccc(F)c(C(=O)c2c[nH]c3ncc(-c4cnc(C5CC5)nc4)cc23)c1F]

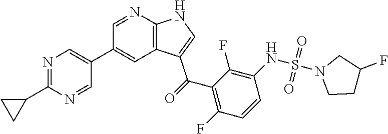 [SMILES: O=C(c1c(F)ccc(NS(=O)(=O)N2CCC(F)C2)c1F)c1c[nH]c2ncc(-c3cnc(C4CC4)nc3)cc12]

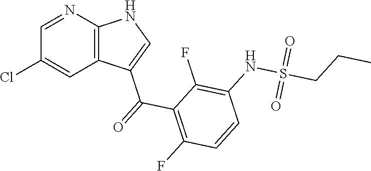 [SMILES: CCCS(=O)(=O)Nc1ccc(F)c(C(=O)c2c[nH]c3ncc(Cl)cc23)c1F]

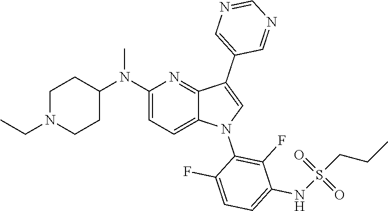 [SMILES: CCCS(=O)(=O)Nc1ccc(F)c(-n2cc(-c3cncnc3)c3nc(N(C)C4CCN(CC)CC4)ccc32)c1F]

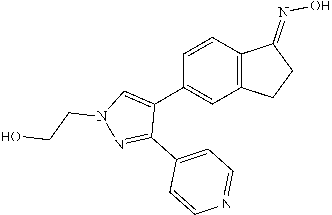 [SMILES: OCCn1cc(-c2ccc3c(c2)CC/C3=N\O)c(-c2ccncc2)n1]

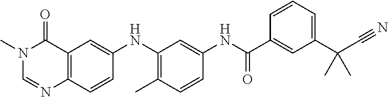 [SMILES: Cc1ccc(NC(=O)c2cccc(C(C)(C)C#N)c2)cc1Nc1ccc2ncn(C)c(=O)c2c1]

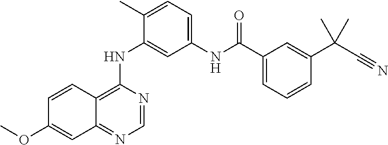 [SMILES: COc1ccc2c(Nc3cc(NC(=O)c4cccc(C(C)(C)C#N)c4)ccc3C)ncnc2c1]

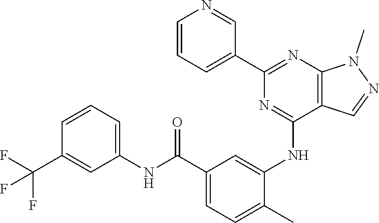 [SMILES: Cc1ccc(C(=O)Nc2cccc(C(F)(F)F)c2)cc1Nc1nc(-c2cccnc2)nc2c1cnn2C]

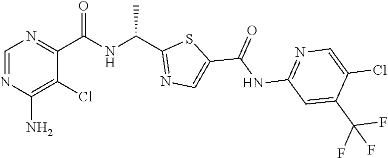 [SMILES: C[C@@H](NC(=O)c1ncnc(N)c1Cl)c1ncc(C(=O)Nc2cc(C(F)(F)F)c(Cl)cn2)s1]

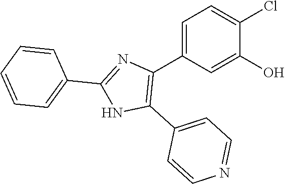 [SMILES: Oc1cc(-c2nc(-c3ccccc3)[nH]c2-c2ccncc2)ccc1Cl]

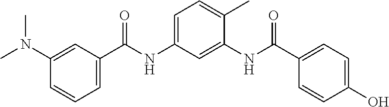 [SMILES: Cc1ccc(NC(=O)c2cccc(N(C)C)c2)cc1NC(=O)c1ccc(O)cc1]

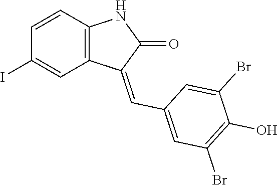 [SMILES: O=C1Nc2ccc(I)cc2/C1=C/c1cc(Br)c(O)c(Br)c1]

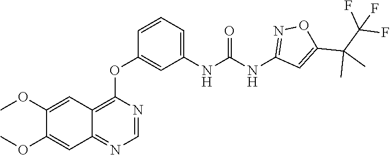 [SMILES: COc1cc2ncnc(Oc3cccc(NC(=O)Nc4cc(C(C)(C)C(F)(F)F)on4)c3)c2cc1OC]

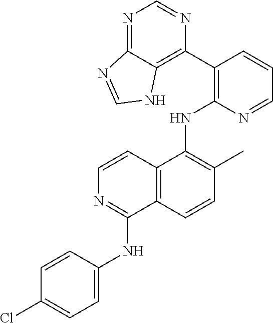 [SMILES: Cc1ccc2c(Nc3ccc(Cl)cc3)nccc2c1Nc1ncccc1-c1ncnc2nc[nH]c12]

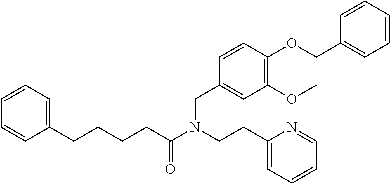 [SMILES: COc1cc(CN(CCc2ccccn2)C(=O)CCCCc2ccccc2)ccc1OCc1ccccc1]

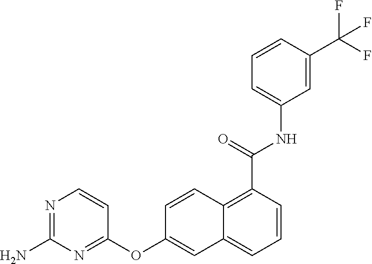 [SMILES: Nc1nccc(Oc2ccc3c(C(=O)Nc4cccc(C(F)(F)F)c4)cccc3c2)n1]

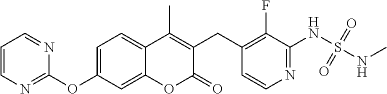 [SMILES: CNS(=O)(=O)Nc1nccc(Cc2c(C)c3ccc(Oc4ncccn4)cc3oc2=O)c1F]

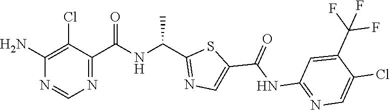 [SMILES: C[C@@H](NC(=O)c1ncnc(N)c1Cl)c1ncc(C(=O)Nc2cc(C(F)(F)F)c(Cl)cn2)s1]

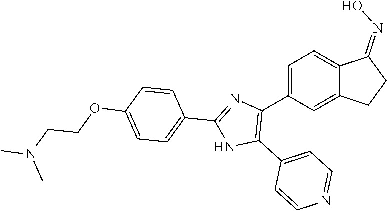 [SMILES: CN(C)CCOc1ccc(-c2nc(-c3ccc4c(c3)CC/C4=N/O)c(-c3ccncc3)[nH]2)cc1]

In some embodiments, the Raf kinase inhibitor is Compound A or a pharmaceutically acceptable salt thereof. In some embodiments, the Raf kinase inhibitor is

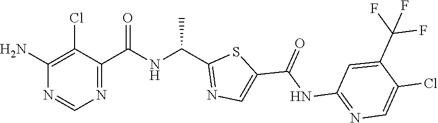 [SMILES: C[C@@H](NC(=O)c1ncnc(N)c1Cl)c1ncc(C(=O)Nc2cc(C(F)(F)F)c(Cl)cn2)s1]

or a pharmaceutically acceptable salt thereof.

### B. Gene Fusions

Described herein are methods of treating a disease or condition associated with a gene fusion. The disease or condition can be a cancer. In some embodiments, the cancer is associated with a gene fusion. In some embodiments, the gene fusion is tumor specific. In some embodiments, the gene fusion is common to several cancers. In some embodiments, the method is used to treat a genetically defined subset of cancer. In some embodiments, the genetically defined subset of cancer comprises one or more gene fusions. In some embodiments, the method comprises administering a compound of Table 1, or a pharmaceutically acceptable salt thereof to a subject having the disease or condition. In some embodiments, the cancer has one more of the following gene fusions: KIAA1549: BRAF, AGK: BRAF, STARD3NL: BRAF, BCAS1: BRAF, KHDRBS2: BRAF, CCDC6: BRAF, FAM131B: BRAF, SRGAP: BRAF, CLCN6: BRAF, GNAI1: BRAF, MRKN1: BRAF, GIT2: BRAF, GTF2I: BRAF, FXR1: BRAF, RNF130: BRAF, MACF1: BRAF, TMEM106B: BRAF, PPC1CC: BRAF, CUX1: BRAF, CCD6: BRAF, PPP1CC: BRAF, SEPT7: BRAF, PDE10A: BRAF, EPB41L2: BRAF, OSBP: BRAF, DAAMI: BRAF, TEX41: BRAF, FOXN3: BRAF, TRIPP1: BRAF, TOMIL2: BRAF, TMEM106B: BRAF, SRGAP3: RAF1, QK1: RAF1, FYCO: RAF1, ATG7: RAF1, NFIA-RAF1, TMF1: RAF1, GOLGA3: RAF1, SOX6: RAF1, BMPRIA: RAF1, PDZRN3: RAF1, SLMAP: RAF1, MAP4: RAF1, BCL6-RAF1, SEPT17: BRAF, ZNF767: BRAF, CCDC91: BRAF, DYNC1/2: BRAF, ZKSCAN1: BRAF, GTF2I: BRAF, MZT1: BRAF, RAD18: BRAF, CUX1: BRAF, CUX1-RAF1, CUL1: BRAF, SLC12A7: BRAF, TRIM24: BRAF, AGAP3: BRAF, AKAP9: BRAF, TAXIBP1: BRAF, CDC27: BRAF, FKBP15: BRAF, SKAP2: BRAF, TARDBP: BRAF, SEPT3: BRAF, ARMC10: BRAF, PAPSS1: BRAF, FCHSD1: BRAF, ERC1: BRAF, CDK5RAP2: BRAF, TMEM178B: BRAF, BAIAP2L1: BRAF, CEP89: BRAF, CNTNAP2: BRAF, EML4: BRAF, KCTD7: BRAF, LSM14A: BRAF, NFIC: BRAF, NUDCD3: BRAF, PHTF2: BRAF, PLIN3: BRAF, RP2: BRAF, SOX5: BRAF, SOX6: BRAF, TLK2: BRAF, ZKSCAN5: BRAF, KLC1-RAF1, DAAMI-RAF1, ZNF444-RAF1, LRCH3-RAF1, GOLGA4-RAF1, CTDSPL-RAF1, PRKAR2A-RAF1, CTNNA1-RAF1, MKRN1-RAF1, DYNCIH1-RAF1, GPHN-RAF1, KLHL7: BRAF, TANK: BRAF, RBMS3: BRAF, FAM114A2: BRAF, AGGF1-RAF1, EPS15: BRAF, NUP214: BRAF, BTF3L4: BRAF, GHR: BRAF, DOCK4: BRAF, ZC3HAV1: BRAF, MKRN1: BRAF, MYRIP: BRAF, SND1: BRAF, TNS3: BRAF, ATG7: BRAF, NUB1: BRAF, STRN3: BRAF, STK35: BRAF, ETFA: BRAF, SVOPL: BRAF, JHDMID: BRAF, PPFIBP2: BRAF, SCL45A3: BRAF, ESRP1-RAF1, AGTRAP: BRAF, SVIP: BRAF, NRF1: BRAF, RAF1-CCDC176, RAF1-TRAK1, ESYT2: BRAF, PCBP2: BRAF, or SALL2: BRAF. In some embodiments, the gene fusion is KIAA1549: BRAF. In some embodiments, the gene fusion is SRGAP3: RAF1. In some embodiments, the gene fusion is SNX8: BRAF.

In one aspect, described herein is a method of treating a disease or condition, such as cancer, associated with a BRAF gene fusion. In some embodiments, the cancer comprises a BRAF gene fusion. In some embodiments, the cancer comprises a wild-type BRAF gene fusion. In some embodiments, the gene fusion is SNX8: BRAF. In some embodiments, the gene fusion is AGK: BRAF. In some embodiments, the gene fusion is STARD3NL: BRAF. In some embodiments, the gene fusion is BCAS1: BRAF. In some embodiments, the gene fusion is KHDRBS2: BRAF. In some embodiments, the gene fusion is CCDC6: BRAF. In some embodiments, the gene fusion is FAM131B: BRAF. In some embodiments, the gene fusion is SRGAP: BRAF. In some embodiments, the gene fusion is CLCN6: BRAF. In some embodiments, the gene fusion is GNAI1: BRAF. In some embodiments, the gene fusion is MRKN1: BRAF. In some embodiments, the gene fusion is GIT2: BRAF. In some embodiments, the gene fusion is GTF2I: BRAF. In some embodiments, the gene fusion is FXR1: BRAF. In some embodiments, the gene fusion is RNF130: BRAF. In some embodiments, the gene fusion is MACF1: BRAF. In some embodiments, the gene fusion is TMEM106B: BRAF. In some embodiments, the gene fusion is PPC1CC: BRAF. In some embodiments, the gene fusion is CUX1: BRAF. In some embodiments, the gene fusion is CCD6: BRAF. In some embodiments, the gene fusion is PPP1CC: BRAF. In some embodiments, the gene fusion is SEPT7: BRAF. In some embodiments, the gene fusion is PDE10A: BRAF. In some embodiments, the gene fusion is EPB41L2: BRAF. In some embodiments, the gene fusion is OSBP: BRAF. In some embodiments, the gene fusion is DAAMI: BRAF. In some embodiments, the gene fusion is TEX41: BRAF. In some embodiments, the gene fusion is FOXN3: BRAF. In some embodiments, the gene fusion is TRIPP1: BRAF. In some embodiments, the gene fusion is TOMIL2: BRAF. In some embodiments, the gene fusion is 5 BRAF fusions. In some embodiments, the gene fusion is TMEM106B: BRAF. In some embodiments, the gene fusion is SEPT17: BRAF. In some embodiments, the gene fusion is ZNF767: BRAF. In some embodiments, the gene fusion is CCDC91: BRAF. In some embodiments, the gene fusion is DYNC1/2: BRAF. In some embodiments, the gene fusion is ZKSCAN1: BRAF. In some embodiments, the gene fusion is GTF2I: BRAF. In some embodiments, the gene fusion is MZT1: BRAF. In some embodiments, the gene fusion is RAD18: BRAF. In some embodiments, the gene fusion is CUX1: BRAF. In some embodiments, the gene fusion is CUL1: BRAF. In some embodiments, the gene fusion is SLC12A7: BRAF. In some embodiments, the gene fusion is TRIM24: BRAF. In some embodiments, the gene fusion is AGAP3: BRAF. In some embodiments, the gene fusion is AKAP9: BRAF. In some embodiments, the gene fusion is TAXIBP1: BRAF. In some embodiments, the gene fusion is CDC27: BRAF. In some embodiments, the gene fusion is FKBP15: BRAF. In some embodiments, the gene fusion is SKAP2: BRAF. In some embodiments, the gene fusion is TARDBP: BRAF. In some embodiments, the gene fusion is SEPT3: BRAF. In some embodiments, the gene fusion is ARMC10: BRAF. In some embodiments, the gene fusion is PAPSS1: BRAF. In some embodiments, the gene fusion is FCHSD1: BRAF. In some embodiments, the gene fusion is ERC1: BRAF. In some embodiments, the gene fusion is CDK5RAP2: BRAF. In some embodiments, the gene fusion is TMEM178B: BRAF. In some embodiments, the gene fusion is BAIAP2L1: BRAF. In some embodiments, the gene fusion is CEP89: BRAF. In some embodiments, the gene fusion is CNTNAP2: BRAF. In some embodiments, the gene fusion is EML4: BRAF. In some embodiments, the gene fusion is KCTD7: BRAF. In some embodiments, the gene fusion is LSM14A: BRAF. In some embodiments, the gene fusion is NFIC: BRAF. In some embodiments, the gene fusion is NUDCD3: BRAF. In some embodiments, the gene fusion is PHTF2: BRAF. In some embodiments, the gene fusion is PLIN3: BRAF. In some embodiments, the gene fusion is RP2: BRAF. In some embodiments, the gene fusion is SOX5: BRAF. In some embodiments, the gene fusion is SOX6: BRAF. In some embodiments, the gene fusion is TLK2: BRAF. In some embodiments, the gene fusion is ZKSCAN5: BRAF. In some embodiments, the gene fusion is KLHL7: BRAF. In some embodiments, the gene fusion is TANK: BRAF. In some embodiments, the gene fusion is RBMS3: BRAF. In some embodiments, the gene fusion is FAM114A2: BRAF. In some embodiments, the gene fusion is EPS15: BRAF. In some embodiments, the gene fusion is NUP214: BRAF. In some embodiments, the gene fusion is BTF3L4: BRAF. In some embodiments, the gene fusion is GHR: BRAF. In some embodiments, the gene fusion is DOCK4: BRAF. In some embodiments, the gene fusion is ZC3HAV1: BRAF. In some embodiments, the gene fusion is MKRN1: BRAF. In some embodiments, the gene fusion is MYRIP: BRAF. In some embodiments, the gene fusion is SND1: BRAF. In some embodiments, the gene fusion is TNS3: BRAF. In some embodiments, the gene fusion is ATG7: BRAF. In some embodiments, the gene fusion is NUB1: BRAF. In some embodiments, the gene fusion is STRN3: BRAF. In some embodiments, the gene fusion is STK35: BRAF. In some embodiments, the gene fusion is ETFA: BRAF. In some embodiments, the gene fusion is SVOPL: BRAF. In some embodiments, the gene fusion is JHDMID: BRAF. In some embodiments, the gene fusion is PPFIBP2: BRAF. In some embodiments, the gene fusion is SCL45A3: BRAF. In some embodiments, the gene fusion is AGTRAP: BRAF. In some embodiments, the gene fusion is SVIP: BRAF. In some embodiments, the gene fusion is NRF1: BRAF. In some embodiments, the gene fusion is ESYT2: BRAF. In some embodiments, the gene fusion is PCBP2: BRAF. In some embodiments, the gene fusion is SALL2: BRAF. In some embodiments, the gene fusion is RIN2: BRAF. In some embodiments, the method comprises administering a compound of Table 1, or a pharmaceutically acceptable salt thereof to a subject having the gene fusion. In some embodiments, the method comprises administering Compound A, or a pharmaceutically acceptable salt thereof to a subject having the gene fusion. In some embodiments, the method comprises administering Compound A to a subject having the gene fusion.

In one aspect, described herein is a method of treating a disease or condition, such as cancer, associated with a CRAF gene fusion. In some embodiments, the cancer comprises a CRAF gene fusion. In some embodiments, the cancer comprises a wild-type CRAF gene fusion. In some embodiments, the gene fusion is SRGAP3: RAF1. In some embodiments, the gene fusion is QK1: RAF1. In some embodiments, the gene fusion is FYCO: RAF1. In some embodiments, the gene fusion is ATG7: RAF1. In some embodiments, the gene fusion is NFIA: RAF1. In some embodiments, the gene fusion is TMF1: RAF1. In some embodiments, the gene fusion is GOLGA3: RAF1. In some embodiments, the gene fusion is SOX6: RAF1. In some embodiments, the gene fusion is BMPRIA: RAF1. In some embodiments, the gene fusion is PDZRN3: RAF1. In some embodiments, the gene fusion is SLMAP: RAF1. In some embodiments, the gene fusion is MAP4: RAF1. In some embodiments, the gene fusion is BCL6: RAF1. In some embodiments, the gene fusion is CUX1: RAF1. In some embodiments, the gene fusion is KLC1: RAF. In some embodiments, the gene fusion is DAAMI: RAF1. In some embodiments, the gene fusion is ZNF444: RAF1. In some embodiments, the gene fusion is LRCH3: RAF1. In some embodiments, the gene fusion is GOLGA4: RAF1. In some embodiments, the gene fusion is CTDSPL: RAF1. In some embodiments, the gene fusion is PRKAR2A: RAF1. In some embodiments, the gene fusion is CTNNA1: RAF1. In some embodiments, the gene fusion is MKRN1: RAF1. In some embodiments, the gene fusion is DYNC1H1: RAF1. In some embodiments, the gene fusion is AGGF1: RAF1. In some embodiments, the gene fusion is ESRP1: RAF1. In some embodiments, the gene fusion is GPHN: RAF1. In some embodiments, the gene fusion is RAF1-CCDC176. In some embodiments, the gene fusion is RAF1-TRAK1. In some embodiments, the method comprises administering a compound of Table 1, or a pharmaceutically acceptable salt thereof to a subject having the gene fusion. In some embodiments, the method comprises administering Compound A, or a pharmaceutically acceptable salt thereof to a subject having the gene fusion. In some embodiments, the method comprises administering Compound A to a subject having the gene fusion.

Exemplar gene fusion of the present disclosure is provided in Table 2.

TABLE 2
Exemplar Gene Fusion

| Gene Fusion | Gene Fusion | Gene Fusion |
|---|---|---|
| KIAA1549:BRAF | FCHSD1:BRAF | BCL6-RAF1 |
| AGK:BRAF | ERC1:BRAF | SEPT17:BRAF |
| STARD3NL:BRAF | CDK5RAP2:BRAF | ZNF767:BRAF |
| BCAS1:BRAF | TMEM178B:BRAF | CCDC91:BRAF |
| KHDRBS2:BRAF | BAIAP2L1:BRAF | DYNC1/2:BRAF |
| CCDC6:BRAF | CEP89:BRAF | ZKSCAN1:BRAF |
| FAM131B:BRAF | CNTNAP2:BRAF | GTF2I:BRAF |
| SRGAP:BRAF | EML4:BRAF | MZT1:BRAF |
| CLCN6:BRAF | KCTD7:BRAF | RAD18:BRAF |
| GNAI1:BRAF | LSM14A:BRAF | CUX1:BRAF |
| MRKN1:BRAF | NFIC:BRAF | CUX1-RAF1 |
| GIT2:BRAF | NUDCD3:BRAF | CUL1:BRAF |
| GTF2I:BRAF | PHTF2:BRAF | SLC12A7:BRAF |
| FXR1:BRAF | PLIN3:BRAF | TRIM24:BRAF |
| RNF130:BRAF | RP2:BRAF | AGAP3:BRAF |
| MACF1:BRAF | SOX5:BRAF | AKAP9:BRAF |
| TMEM106B:BRAF | SOX6:BRAF | TAX1BP1:BRAF |
| PPC1CC:BRAF | TLK2:BRAF | CDC27:BRAF |
| CUX1:BRAF | ZKSCAN5:BRAF | FKBP15:BRAF |
| CCD6:BRAF | KLC1-RAF1 | SKAP2:BRAF |
| PPP1CC:BRAF | DAAM1-RAF1 | TARDBP:BRAF |
| SEPT7:BRAF | ZNF444-RAF1 | SEPT3:BRAF |
| PDE10A:BRAF | LRCH3-RAF1 | TNS3:BRAF |
| EPB41L2:BRAF | GOLGA4-RAF1 | ATG7:BRAF |
| OSBP:BRAF | CTDSPL-RAF1 | NUB1:BRAF |
| DAAM1:BRAF | PRKAR2A-RAF1 | STRN3:BRAF |
| TEX41:BRAF | CTNNA1-RAF1 | STK35:BRAF |
| FOXN3:BRAF | MKRN1-RAF1 | ETFA:BRAF |
| TRIPP1:BRAF | DYNC1H1-RAF1 | SVOPL:BRAF |
| TOM1L2:BRAF | GPHN-RAF1 | JHDM1D:BRAF |
| TMEM106B:BRAF | KLHL7:BRAF | PPFIBP2:BRAF |
| SRGAP3:RAF1 | TANK:BRAF | SCL45A3:BRAF |
| QK1:RAF1 | RBMS3:BRAF | ESRP1-RAF1 |
| FYCO:RAF1 | FAM114A2:BRAF | AGTRAP:BRAF |
| ATG7:RAF1 | AGGF1-RAF1 | SVIP:BRAF |
| NFIA-RAF1 | EPS15:BRAF | NRF1:BRAF |
| TMF1:RAF1 | NUP214:BRAF | RAF1-CCDC176 |
| GOLGA3:RAF1 | BTF3L4:BRAF | RAF1-TRAK1 |
| SOX6:RAF1 | GHR:BRAF | ESYT2:BRAF |
| BMPR1A:RAF1 | DOCK4:BRAF | PCBP2:BRAF |
| PDZRN3:RAF1 | ZC3HAV1:BRAF | SALL2:BRAF |
| SLMAP:RAF1 | MKRN1:BRAF | ARMC10:BRAF |
| MAP4:RAF1 | MYRIP:BRAF | PAPSS1:BRAF |
| SNX8:BRAF | SND1:BRAF | RIN2:BRAF |

### C. Treatment of Solid Tumors

In one aspect, described herein are methods of treating a disease or condition. In some embodiments, described herein are methods of treating a cancer in a subject in need thereof. In some embodiments, described herein are methods of treating solid tumors. In some embodiments, the cancer is a solid tumor. In some embodiments, the solid tumor is an extracranial tumor. In some embodiments, the solid tumor is an area other than the brain (e.g., sarcoma, acinar pancreas cancer, and the like). In some embodiments, the solid tumor is a bone sarcoma or a soft tissue sarcoma. In some embodiments, the solid tumor is a soft tissue sarcoma. In some embodiments, the soft tissue sarcoma is selected from: fibrosarcoma, Malignant fibrous histiocytoma, Dermatofibrosarcoma, Liposarcoma, Rhabdomyosarcoma, Leiomyosarcoma, Hemangiosarcoma, Kaposi's sarcoma, Lymphangiosarcoma, Synovial sarcoma, Neurofibrosarcoma, Extraskeletal chondrosarcoma, and Extraskeletal osteosarcoma. In some embodiments, the method comprises administering a compound of Table 1 or a pharmaceutically acceptable salt thereof to a subject in need thereof. In some embodiments, the method comprises administering Compound A or a pharmaceutically acceptable salt thereof to the subject. In some embodiments, the method comprises administering Compound A to the subject.

In some embodiments, the solid tumor is a bone sarcoma. In some embodiments, the bone sarcoma is selected from osteosarcoma, chonrosarcoma, spindle cell sarcoma, hemangioendothelioma, angiosarcoma, fibrosarcoma, chordoma, adamantinoma, liposarcoma, leiomyosarcoma, malignant peripheral nerve sheath tumor, rhabdomyosarcoma, synovial sarcoma, and malignant solitary fibrous tumor. In some embodiments, the bone sarcoma is spindle cell sarcoma.

In some embodiments, the cancer is an advanced solid tumor. In some embodiments, the tumor is an advanced solid tumor. In some embodiments, the cancer is a recurrent cancer. In some embodiments, the solid tumor has received at least one prior therapy before the adminstration of a Raf kinase inhibitor or a pharmaceutically acceptable salt thereof. In some embodiments, the solid tumor has received at least one prior therapy before the adminstration of Compound A. In some embodiments, the prior therapy is chemotherapy therapy, hormone therapy, immunotherapy, or radiation therapy. In some embodiments, the prior therapy is chemotherapy therapy. In some embodiments, the prior therapy is hormone therapy. In some embodiments, the prior therapy is immunotherapy. In some embodiments, the prior therapy is radiation therapy.

In some embodiments, provided herein are methods of treating a tumor, wherein the tumor comprises or is characterized as having a gene fusion. In some embodiments, the method comprises administering a compound of Table 1 (such as Compound A) or a pharmaceutically acceptable salt thereof to a subject in need thereof. In some embodiments, the tumor is a solid tumor. In certain embodiments, the tumor is a tumor selected from the group consisting of: colorectal tumor, pancreatic tumor, lung tumor, ovarian tumor, liver tumor, breast tumor, kidney tumor, prostate tumor, neuroendocrine tumor, gastrointestinal tumor, melanoma, cervical tumor, bladder tumor, glioblastoma, and head and neck tumor. In certain embodiments, the tumor is a colorectal tumor. In certain embodiments, the tumor is an ovarian tumor. In some embodiments, the tumor is a breast tumor. In some embodiments, the tumor is a lung tumor. In certain embodiments, the tumor is a pancreatic tumor. In certain embodiments, the tumor is a melanoma tumor. In some embodiments, the tumor is a solid tumor. In certain embodiments, the cancer is a cancer selected from the group consisting of colorectal cancer, pancreatic cancer, lung cancer, ovarian cancer, liver cancer, breast cancer, kidney cancer, prostate cancer, gastrointestinal cancer, melanoma, cervical cancer, neuroendocrine cancer, bladder cancer, glioblastoma, and head and neck cancer. In certain embodiments, the cancer is pancreatic cancer. In certain embodiments, the cancer is ovarian cancer. In certain embodiments, the cancer is colorectal cancer. In certain embodiments, the cancer is breast cancer. In certain embodiments, the cancer is prostate cancer. In certain embodiments, the cancer is lung cancer. In certain embodiments, the cancer is melanoma. In some embodiments, the cancer is a solid cancer.

The present disclosure provides for methods of treating cancer comprising administering to a subject a therapeutically effective amount of a Raf inhibitor described herein (e.g., a subject in need of treatment). In certain embodiments, the subject is a human. In certain embodiments, the subject has a cancerous tumor. In certain embodiments, the subject has had a tumor at least partially removed. In certain embodiments, the subject has gene fusion described herein.

In some embodiments, the cancer is a hematologic cancer. In some embodiment, the cancer is selected from the group consisting of: acute myelogenous leukemia (AML), Hodgkin lymphoma, multiple myeloma, T cell acute lymphoblastic leukemia (T-ALL), chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), and cutaneous T cell lymphoma (CTCL).

In some embodiments, the Raf inhibitor is administered as a combination therapy. Combination therapy with two or more therapeutic agents can use agents that work by different mechanisms of action, although this is not required. Combination therapy using agents with different mechanisms of action can result in additive or synergetic effects. Combination therapy can allow for a lower dose of each agent than is used in monotherapy, thereby reducing toxic side effects and/or increasing the therapeutic index of the agent(s).

The methods of the disclosure can be used to treat any suitable cancer known in the art. In some embodiments, the cancer has a gene fusion provided in Table 2. Non-limiting examples of cancers to be treated by the methods of the present disclosure can include melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies. In some embodiments, the cancer is low grade glioma (LGG), e.g., pediatric low-grade glioma (pLGG). In some embodiments, the LGG or pLGG has a gene fusion of Table 2. In some embodiments, the cancer is pLGG with RIN2: BRAF gene fusion. In some embodiments, the cancer is relapsed or refractory. In some embodiments, the subject has received prior treatment before the administration of a Raf inhibitor (such as Compound A or a salt thereof). In some embodiments, the subject has received prior standard-of-care treatment before the administration of a Raf inhibitor (such as Compound A or a salt thereof). In some embodiments, the subject has not received prior treatment before the administration of a Raf inhibitor (such as Compound A or a salt thereof). In some embodiments, a Raf inhibitor described herein (such as Compound A or a salt thereof) is administered as a front-line therapy. In some embodiments, a Raf inhibitor described herein (such as Compound A or a salt thereof) is administered as a second-line therapy.

Additionally, the disease or condition provided herein includes refractory or recurrent malignancies whose growth may be inhibited using the methods of treatment of the present disclosure. In some embodiments, a cancer to be treated by the methods of treatment of the present disclosure is selected from the group consisting of carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, and combinations thereof. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, a cancer to be treated by the methods of the present disclosure further include sarcomata (for example, myogenic sarcoma), leukosis, neuroma, melanoma, and lymphoma. In some embodiments, a cancer to be treated by the methods of the present disclosure is breast cancer. In some embodiments, a cancer to be treated by the methods of treatment of the present disclosure is triple negative breast cancer (TNBC). In some embodiments, a cancer to be treated by the methods of treatment of the present disclosure is ovarian cancer. In some embodiments, a cancer to be treated by the methods of treatment of the present disclosure is colorectal cancer. In some embodiments, the cancer has a gene fusion provided in Table 2.

In some embodiments, a patient or population of patients to be treated with a pharmaceutical composition of the present disclosure have a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma. In some embodiments, a patient or population of patients to be treated with a pharmaceutical composition of the present disclosure have a hematological cancer. In some embodiments, the patient has a hematological cancer such as Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), or Multiple myeloma (“MM”). In some embodiments, a patient or population of patients to be treated having the cancer selected from the group consisting of ovarian cancer, lung cancer and melanoma. In some embodiments, the cancer has a gene fusion provided in Table 2.

Specific examples of cancers that can be prevented and/or treated in accordance with present disclosure include, but are not limited to, the following: renal cancer, kidney cancer, glioblastoma multiforme, metastatic breast cancer; breast carcinoma; breast sarcoma; neurofibroma; neurofibromatosis; pediatric tumors; neuroblastoma; malignant melanoma; carcinomas of the epidermis; leukemias such as but not limited to, acute leukemia, acute lymphocytic leukemia, acute myelocytic leukemias such as myeloblastic, promyelocytic, myelomonocytic, monocytic, erythroleukemia leukemias and myclodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; cervical carcinoma; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; colorectal cancer, KRAS mutated colorectal cancer; colon carcinoma; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as KRAS-mutated non-small cell lung cancer, non-small cell lung cancer, squamous cell carcinoma (epidermoid carcinoma), adenocarcinoma, large-cell carcinoma and small-cell lung cancer; lung carcinoma; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, androgen-independent prostate cancer, androgen-dependent prostate cancer, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acrallentiginous melanoma; kidney cancers such as but not limited to renal cell cancer, adenocarcinoma, hypernephroma, fibrosarcoma, transitional cell cancer (renal pelvis and/or uterer); renal carcinoma; Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas. In some embodiments, the cancer has a gene fusion provided in Table 2.

### D. Methods of Treating Cancer

Provided herein are methods of treating cancers that harbor one or more gene fusions. In some aspects, the method of treating cancer comprises administering a Raf kinase inhibitor to a subject in need thereof, wherein the Raf inhibitor is selected from a compound of Table 1, or a pharmaceutically acceptable salt thereof, and wherein the subject has a cancer harboring a gene fusion. In some embodiments, the gene fusion is selected from Table 2.

In some aspects, the method of treating cancer comprises administering a Raf kinase inhibitor to a subject in need thereof, wherein the subject has a CRAF gene fusion, wherein the Raf kinase inhibitor is (R)-2-(1-(6-amino-5-chloropyrimidine-4-carboxamido)ethyl)-N-(5-chloro-4-(trifluoromethyl) pyridin-2-yl) thiazole-5-carboxamide (Compound A) or a pharmaceutically acceptable salt thereof. In some embodiments, the CRAF fusion is a wild-type CRAF fusion. In some embodiments, the CRAF gene fusion is SRGAP3: RAF1, QK1: RAF1, FYCO: RAF1, ATG7: RAF1, NFIA: RAF1, TMF1: RAF1, GOLGA3: RAF1, SOX6: RAF1, BMPRIA: RAF1, PDZRN3: RAF1, SLMAP: RAF1, MAP4: RAF1, BCL6: RAF1, CUX1: RAF1, KLC1: RAF1, DAAMI: RAF1, ZNF444: RAF1, LRCH3: RAF1, GOLGA4: RAF1, CTDSPL: RAF1, PRKAR2A: RAF1, CTNNA1: RAF1, MKRN1: RAF1, DYNCIH1: RAF1, AGGF1: RAF1, ESRP1: RAF1, GPHN: RAF1, RAF1-CCDC176, or RAF1-TRAK1.

In some aspects, the method of treating cancer, the method comprising administering a Raf kinase inhibitor to a subject in need thereof, wherein the subject has one or more of the following gene fusions: KIAA1549: BRAF, AGK: BRAF, STARD3NL: BRAF, BCAS1: BRAF, KHDRBS2: BRAF, CCDC6: BRAF, FAM131B: BRAF, SRGAP: BRAF, CLCN6: BRAF, GNAI1: BRAF, MRKN1: BRAF, GIT2: BRAF, GTF2I: BRAF, FXR1: BRAF, RNF130: BRAF, MACF1: BRAF, TMEM106B: BRAF, PPC1CC: BRAF, CUX1: BRAF, CCD6: BRAF, PPP1CC: BRAF, SEPT7: BRAF, PDE10A: BRAF, EPB41L2: BRAF, OSBP: BRAF, DAAMI: BRAF, TEX41: BRAF, FOXN3: BRAF, TRIPP1: BRAF, TOMIL2: BRAF, TMEM106B: BRAF, SRGAP3: RAF1, QK1: RAF1, FYCO: RAF1, ATG7: RAF1, NFIA-RAF1, TMF1: RAF1, GOLGA3: RAF1, SOX6: RAF1, BMPRIA: RAF1, PDZRN3: RAF1, SLMAP: RAF1, MAP4: RAF1, BCL6-RAF1, SEPT17: BRAF, ZNF767: BRAF, CCDC91: BRAF, DYNC1/2: BRAF, ZKSCAN1: BRAF, GTF2I: BRAF, MZT1: BRAF, RAD18: BRAF, CUX1: BRAF, CUX1-RAF1, CUL1: BRAF, SLC12A7: BRAF, TRIM24: BRAF, AGAP3: BRAF, AKAP9: BRAF, TAXIBP1: BRAF, CDC27: BRAF, FKBP15: BRAF, SKAP2: BRAF, TARDBP: BRAF, SEPT3: BRAF, ARMC10: BRAF, PAPSS1: BRAF, FCHSD1: BRAF, ERC1: BRAF, CDK5RAP2: BRAF, TMEM178B: BRAF, BAIAP2L1: BRAF, CEP89: BRAF, CNTNAP2: BRAF, EML4: BRAF, KCTD7: BRAF, LSM14A: BRAF, NFIC: BRAF, NUDCD3: BRAF, PHTF2: BRAF, PLIN3: BRAF, RP2: BRAF, SOX5: BRAF, SOX6: BRAF, TLK2: BRAF, ZKSCAN5: BRAF, KLC1-RAF1, DAAMI-RAF1, ZNF444-RAF1, LRCH3-RAF1, GOLGA4-RAF1, CTDSPL-RAF1, PRKAR2A-RAF1, CTNNA1-RAF1, MKRN1-RAF1, DYNCIH1-RAF1, GPHN-RAF1, KLHL7: BRAF, TANK: BRAF, RBMS3: BRAF, FAM114A2: BRAF, AGGF1-RAF1, EPS15: BRAF, NUP214: BRAF, BTF3L4: BRAF, GHR: BRAF, DOCK4: BRAF, ZC3HAV1: BRAF, MKRN1: BRAF, MYRIP: BRAF, SND1: BRAF, TNS3: BRAF, ATG7: BRAF, NUB1: BRAF, STRN3: BRAF, STK35: BRAF, ETFA: BRAF, SVOPL: BRAF, JHDMID: BRAF, PPFIBP2: BRAF, SCL45A3: BRAF, ESRP1-RAF1, AGTRAP: BRAF, SVIP: BRAF, NRF1: BRAF, RAF1-CCDC176, RAF1-TRAK1, ESYT2: BRAF, PCBP2: BRAF, and SALL2: BRAF, wherein the Raf kinase inhibitor is (R)-2-(1-(6-amino-5-chloropyrimidine-4-carboxamido)ethyl)-N-(5-chloro-4-(trifluoromethyl) pyridin-2-yl) thiazole-5-carboxamide (Compound A) or a pharmaceutically acceptable salt thereof, and wherein the subject is at least 18 years of age. In some embodiments, the subject is less than 18 years of age.

In some aspects, the method disclosed herein is a method of treating a solid tumor in an adult subject, the method comprising administering a Raf kinase inhibitor to a subject in need thereof, wherein the subject has a CRAF gene fusion, a BRAF gene fusion, or both, wherein the Raf kinase inhibitor is (R)-2-(1-(6-amino-5-chloropyrimidine-4-carboxamido)ethyl)-N-(5-chloro-4-(trifluoromethyl) pyridin-2-yl) thiazole-5-carboxamide or a pharmaceutically acceptable salt thereof, wherein the subject is at least 18 years of age. In some embodiments, the subject has a wild-type CRAF fusion, wild-type BRAF fusion, or both. In some embodiments, the subject has a gene fusion that is KIAA1549: BRAF or SRGAP3: RAF1.

In some aspects, the method disclosed herein is a method of treating a solid tumor in an adult subject, the method comprising administering a Raf kinase inhibitor to a subject in need thereof, wherein the subject has a wild-type CRAF gene fusion, a wild-type BRAF gene fusion, or both; and wherein the Raf kinase inhibitor is (R)-2-(1-(6-amino-5-chloropyrimidine-4-carboxamido)ethyl)-N-(5-chloro-4-(trifluoromethyl) pyridin-2-yl) thiazole-5-carboxamide (Compound A) or a pharmaceutically acceptable salt thereof.

In some aspects, provided herein is a method of treating cancer, the method comprising administering a Raf kinase inhibitor to a subject in need thereof, wherein the subject has one or more of the following gene fusions: AGK: BRAF, STARD3NL: BRAF, BCAS1: BRAF, KHDRBS2: BRAF, CCDC6: BRAF, FAM131B: BRAF, SRGAP: BRAF, CLCN6: BRAF, GNAI1: BRAF, MRKN1: BRAF, GIT2: BRAF, GTF2I: BRAF, FXR1: BRAF, RNF130: BRAF, MACF1: BRAF, TMEM106B: BRAF, PPC1CC: BRAF, CUX1: BRAF, CCD6: BRAF, PPP1CC: BRAF, SEPT7: BRAF, PDE10A: BRAF, EPB41L2: BRAF, OSBP: BRAF, DAAMI: BRAF, TEX41: BRAF, FOXN3: BRAF, TRIPP1: BRAF, TOMIL2: BRAF, TMEM106B: BRAF, QK1: RAF1, FYCO: RAF1, ATG7: RAF1, NFIA-RAF1, TMF1: RAF1, GOLGA3: RAF1, SOX6: RAF1, BMPRIA: RAF1, PDZRN3: RAF1, SLMAP: RAF1, MAP4: RAF1, BCL6-RAF1, SEPT17: BRAF, ZNF767: BRAF, CCDC91: BRAF, DYNC1/2: BRAF, ZKSCAN1: BRAF, GTF2I: BRAF, MZT1: BRAF, RAD18: BRAF, CUX1: BRAF, CUX1-RAF1, CUL1: BRAF, SLC12A7: BRAF, TRIM24: BRAF, AGAP3: BRAF, AKAP9: BRAF, TAXIBP1: BRAF, CDC27: BRAF, FKBP15: BRAF, SKAP2: BRAF, TARDBP: BRAF, SEPT3: BRAF, ARMC10: BRAF, PAPSS1: BRAF, FCHSD1: BRAF, ERC1: BRAF, CDK5RAP2: BRAF, TMEM178B: BRAF, BAIAP2L1: BRAF, CEP89: BRAF, CNTNAP2: BRAF, EML4: BRAF, KCTD7: BRAF, LSM14A: BRAF, NFIC: BRAF, NUDCD3: BRAF, PHTF2: BRAF, PLIN3: BRAF, RP2: BRAF, SOX5: BRAF, SOX6: BRAF, TLK2: BRAF, ZKSCAN5: BRAF, KLC1-RAF1, DAAMI-RAF1, ZNF444-RAF1, LRCH3-RAF1, GOLGA4-RAF1, CTDSPL-RAF1, PRKAR2A-RAF1, CTNNA1-RAF1, MKRN1-RAF1, DYNCIH1-RAF1, GPHN-RAF1, KLHL7: BRAF, TANK: BRAF, RBMS3: BRAF, FAM114A2: BRAF, AGGF1-RAF1, EPS15: BRAF, NUP214: BRAF, BTF3L4: BRAF, GHR: BRAF, DOCK4: BRAF, ZC3HAV1: BRAF, MKRN1: BRAF, MYRIP: BRAF, SND1: BRAF, TNS3: BRAF, ATG7: BRAF, NUB1: BRAF, STRN3: BRAF, STK35: BRAF, ETFA: BRAF, SVOPL: BRAF, JHDMID: BRAF, PPFIBP2: BRAF, SCL45A3: BRAF, ESRP1-RAF1, AGTRAP: BRAF, SVIP: BRAF, NRF1: BRAF, RAF1-CCDC176, RAF1-TRAK1, ESYT2: BRAF, PCBP2: BRAF, or SALL2: BRAF, and wherein the Raf kinase inhibitor is (R)-2-(1-(6-amino-5-chloropyrimidine-4-carboxamido)ethyl)-N-(5-chloro-4-(trifluoromethyl) pyridin-2-yl) thiazole-5-carboxamide (Compound A) or a pharmaceutically acceptable salt thereof. In some embodiments, the gene fusion is BRAF gene fusion as described herein. In some embodiments, the BRAF gene fusion is a wild-type BRAF gene fusion. In some embodiments, the BRAF fusion is selected from: AGK: BRAF, STARD3NL: BRAF, BCAS1: BRAF, KHDRBS2: BRAF, CCDC6: BRAF, FAM131B: BRAF, SRGAP: BRAF, CLCN6: BRAF, GNAI1: BRAF, MRKN1: BRAF, GIT2: BRAF, GTF2I: BRAF, FXR1: BRAF, RNF130: BRAF, MACF1: BRAF, TMEM106B: BRAF, PPC1CC: BRAF, CUX1: BRAF, CCD6: BRAF, PPP1CC: BRAF, SEPT7: BRAF, PDE10A: BRAF, EPB41L2: BRAF, OSBP: BRAF, DAAMI: BRAF, TEX41: BRAF, FOXN3: BRAF, TRIPP1: BRAF, TOMIL2: BRAF, 5 BRAF fusions, TMEM106B: BRAF, SEPT17: BRAF, ZNF767: BRAF, CCDC91: BRAF, DYNC1/2: BRAF, ZKSCAN1: BRAF, GTF2I: BRAF, MZT1: BRAF, RAD18: BRAF, CUX1: BRAF, CUL1: BRAF, SLC12A7: BRAF, TRIM24: BRAF, AGAP3: BRAF, AKAP9: BRAF, TAXIBP1: BRAF, CDC27: BRAF, FKBP15: BRAF, SKAP2: BRAF, TARDBP: BRAF, SEPT3: BRAF, ARMC10: BRAF, PAPSS1: BRAF, FCHSD1: BRAF, ERC1: BRAF, CDK5RAP2: BRAF, TMEM178B: BRAF, BAIAP2L1: BRAF, CEP89: BRAF, CNTNAP2: BRAF, EML4: BRAF, KCTD7: BRAF, LSM14A: BRAF, NFIC: BRAF, NUDCD3: BRAF, PHTF2: BRAF, PLIN3: BRAF, RP2: BRAF, SOX5: BRAF, SOX6: BRAF, TLK2: BRAF, ZKSCAN5: BRAF, KLHL7: BRAF, TANK: BRAF, RBMS3: BRAF, FAM114A2: BRAF, EPS15: BRAF, NUP214: BRAF, BTF3L4: BRAF, GHR: BRAF, DOCK4: BRAF, ZC3HAV1: BRAF, MKRN1: BRAF, MYRIP: BRAF, SND1: BRAF, TNS3: BRAF, ATG7: BRAF, NUB1: BRAF, STRN3: BRAF, STK35: BRAF, ETFA: BRAF, SVOPL: BRAF, JHDMID: BRAF, PPFIBP2: BRAF, SCL45A3: BRAF, AGTRAP: BRAF, SVIP: BRAF, NRF1: BRAF, ESYT2: BRAF, PCBP2: BRAF, and SALL2: BRAF. In some embodiments, the gene fusion is a RAF1 gene fusion. In some embodiments, the gene fusion is a wild-type gene fusion. In some embodiments, the RAF1 gene fusion is selected from: SRGAP3: RAF1, QK1: RAF1, FYCO: RAF1, ATG7: RAF1, NFIA: RAF1, TMF1: RAF1, GOLGA3: RAF1, SOX6: RAF1, BMPRIA: RAF1, PDZRN3: RAF1, SLMAP: RAF1, MAP4: RAF1, BCL6: RAF1, CUX1: RAF1, KLC1: RAF1, DAAMI: RAF1, ZNF444: RAF1, LRCH3: RAF1, GOLGA4: RAF1, CTDSPL: RAF1, PRKAR2A: RAF1, CTNNA1: RAF1, MKRN1: RAF1, DYNCIH1: RAF1, AGGF1: RAF1, ESRP1: RAF1, GPHN: RAF1, RAF1-CCDC176, and RAF1-TRAK1.

In some embodiments, the method provided herein further comprises obtaining a cancer sample from the subject and subjecting the cancer sample to genomic testing (e.g., mutational testing) prior to the administering of a Raf kinase inhibitor. In some embodiments, the method further comprises identifying the cancer sample as harboring at least one gene fusion as provided herein through genomic testing. In some embodiments, the method comprises identifying the cancer sample as having a CRAF gene fusion or BRAF gene fusion. In some embodiments, the method comprises identifying the cancer sample as having a wild-type CRAF gene fusion or wild-type BRAF gene fusion. In some embodiments, the method comprises identifying the cancer sample as having a CRAF gene fusion. In some embodiments, the method comprises identifying the cancer sample as having a BRAF gene fusion. In some embodiments, the method further comprises obtaining a cancer sample from the subject and subjecting the cancer sample to genomic testing prior to the administering of Compound A. In some embodiments, the genetic testing is genomic profiling. In some embodiments, the genomic profiling is whole exome sequencing.

In some embodiments, a method provided herein further comprises the administration of a dermatological agent. In some embodiments, the dermatological agent is diphenhydramine. In some embodiments, the dermatological agent is administered after the appearance of a rash in the subject. In some embodiments, the dermatological agent is administered before the appearance of a rash in the subject. In some embodiments, the dermatological agent and the Raf kinase inhibitor are administered together.

In some embodiments, the administration occurs in a plurality of treatment stages which alternate between an administration stage and a non-administration stage. In some embodiments, the administration stage has a duration of 28 days. In some embodiments, cancer may be radiographically monitored to evaluate disease progression. In some embodiments, the administration stage comprises the administration of a Raf kinase inhibitor or a pharmaceutically acceptable salt thereof once weekly. In some embodiments, the administration stage comprises the administration of Compound A or a pharmaceutically acceptable salt thereof once weekly.

In some embodiments, the subject in need thereof is from about 6 months to 25 years old. In some embodiments, the subject in need thereof is from about 1 year to 25 years old. In some embodiments, a subject in need thereof is 25 years of age of less. In some embodiments, a subject in need thereof is 20 years of age or less. In some embodiments, a subject in need thereof is 15 years of age or less. In some embodiments, a subject in need thereof is 10 years of age or less. In some embodiments, a subject in need thereof is 20, 19, 18, 17, 16, 15, 14, 13, 12, 11, 10 years of age or less. In some embodiments, the subject in need thereof is 1, 2, 3, 4, 5, 6, 7, 8, 9, 10, 11, 12, 13, 14, 15, 16, 17, 18, 19, 20, 21, 22, 23, 24, 25 years old. In some embodiments, the subject in need thereof is less than 18 years old. In some embodiments, the subject in need thereof is older than 18 years old.

Subjects can be, for example, mammal, humans, pregnant women, elderly adults, adults, adolescents, pre-adolescents, children, toddlers, infants, newborn, or neonates. A subject can be a patient. In some cases, a subject can be a human. In some cases, a subject can be a child (e.g., a young human being below the age of puberty). In some cases, a subject can be an infant. In some cases, the subject can be a formula-fed infant. In some cases, a subject can be an individual enrolled in a clinical study. In some cases, a subject can be a laboratory animal, for example, a mammal, or a rodent. In some cases, the subject can be a mouse. In some cases, the subject can be an obese or overweight subject.

In some embodiments, the subject has previously been treated with one or more different cancer treatment modalities. In some embodiments, the subject has previously been treated with one or more of radiotherapy, chemotherapy, or immunotherapy. In some embodiments, the subject has been treated with one, two, three, four, or five lines of prior therapy. In some embodiments, the prior therapy is a cytotoxic therapy.

In some embodiments, a Raf Inhibitor or a pharmaceutically salt thereof, is administered at 420 mg/m². In some embodiments, Compound A or a pharmaceutically acceptable salt thereof is administered at 420 mg/m².

In some embodiments, a Raf Inhibitor or a pharmaceutically salt thereof, comprises an initial dose of the Raf Inhibitor or a pharmaceutically acceptable salt thereof equivalent to about 280 mg/m² to about 600 mg/m² of the Raf Inhibitor per week. In some embodiments, wherein the administering of Compound A or a pharmaceutically acceptable salt thereof, comprises an initial dose of the Compound A or a pharmaceutically acceptable salt thereof equivalent to about 280 mg/m² to about 600 mg/m² of Compound A per week.

In some embodiments, a Raf inhibitor or a pharmaceutically acceptable salt is administered in an amount of up to about 100 mg, 200 mg, 300 mg, 400 mg, 500 mg, about 600 mg, about 700 mg, about 800 mg, about 900 mg, or about 1000 mg of per dose. In some embodiments, the Raf inhibitor or a pharmaceutically acceptable salt is administered in an amount of up to about 800 mg per dose. In some embodiments, the Raf inhibitor or a pharmaceutically acceptable salt thereof is administered in an amount of about 100 mg to about 800 mg per dose. In some embodiments, the Raf inhibitor or a pharmaceutically acceptable salt thereof is administered in an amount of about 50 mg to about 1000 mg per dose. In some embodiments, the Raf inhibitor or a pharmaceutically acceptable salt thereof is administered in an amount of about 50 mg to about 200 mg per dose. In some embodiments, the Raf inhibitor or a pharmaceutically acceptable salt thereof is administered in an amount of about 500 mg to about 800 mg per dose. In some embodiments, the Raf inhibitor or a pharmaceutically acceptable salt thereof is administered in an amount of about 100 mg to about 500 mg per dose. In some embodiments, the Raf inhibitor or a pharmaceutically acceptable salt thereof is administered in an amount of about 400 mg to about 700 mg per dose. In some embodiments, the Raf inhibitor or a pharmaceutically acceptable salt is administered in an amount of up to about 600 mg per dose. In some embodiments, the Raf inhibitor or a pharmaceutically acceptable salt is administered in an amount of up to about 400 mg per dose. In some embodiments, the Raf inhibitor or a pharmaceutically acceptable salt is administered in an amount of up to about 200 mg per dose. In some embodiments, the suitable dosage of the Raf inhibitor, or a pharmaceutically acceptable salt is from about 100 mg to about 1000 mg per dose. In some embodiments, suitable dosages are about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, about 500 mg, about 520 mg, about 530 mg, about 560 mg, about 580 mg, about 600 mg, about 620 mg, about 640 mg, about 660 mg, about 680 mg, about 700 mg, about 720 mg, about 740 mg, about 760 mg, about 780 mg, about 800 mg per dose, about 820 mg per dose, about 840 mg per dose, about 860 mg per dose, about 880 mg per dose, about 900 mg per dose, about 920 mg per dose, about 940 mg per dose, about 960 mg per dose, about 980 mg per dose, or about 1000 mg per dose. In some embodiments, the Raf inhibitor or a pharmaceutically acceptable salt thereof is administered once a month. In some embodiments, the Raf inhibitor or a pharmaceutically acceptable salt thereof is administered once a week. In some embodiments, the Raf inhibitor or a pharmaceutically acceptable salt thereof is administered 2, 3, 4, 5, 6, or 7 times a week.

In some embodiments, the Raf inhibitor or a pharmaceutically acceptable salt thereof can be administered to a subject in about 100 to about 1000 mg/m² per week. In some embodiments, the starting dose of the Raf inhibitor is about 20 mg/m², about 25 mg/m², about 30 mg/m², about 35 mg/m², about 40 mg/m², about 45 mg/m², about 50 mg/m², about 55 mg/m², about 60 mg, about 65 mg/m², about 70 mg/m², about 75 mg/m², about 80 mg/m², about 85 mg/m², about 90 mg/m², about 95 mg/m², about 100 mg/m², about 105 mg/m², about 110 mg/m², about 115 mg/m², about 120 mg/m², about 125 mg/m², about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², about 200 mg/m², about 220 mg/m² about 240 mg/m², about 260 mg/m², about 280 mg/m², about 300 mg/m², about 320 mg/m², about 340 mg/m², about 360 mg/m², about 380 mg/m², about 400 mg/m², about 420 mg/m², about 440 mg/m², about 460 mg/m², about 480 mg/m², about 500 mg/m², about 520 mg/m² about 530 mg/m², about 560 mg/m², about 580 mg/m², about 600 mg/m², about 620 mg/m², about 640 mg/m², about 660 mg/m², about 680 mg/m², about 700 mg/m², about 720 mg/m², about 740 mg/m², about 760 mg/m², about 780 mg/m², about 800 mg/m², about 825 mg/m², about 850 mg/m², about 875 mg/m², about 900 mg/m², about 925 mg/m², about 950 mg/m², about 975 mg/m², or about 1000 mg/m² per week. In some embodiments, the Raf inhibitor or a pharmaceutically acceptable salt thereof is administered once a week. In some embodiments, the Raf inhibitor or a pharmaceutically acceptable salt thereof is administered 2, 3, 4, 5, 6, or 7 times a week. In some embodiments, the dose of the Raf inhibitor or a pharmaceutically acceptable salt thereof is about 825 mg/m² per week. In some embodiments, the dose of the Raf inhibitor or a pharmaceutically acceptable salt thereof is about 660 mg/m² per week. In some embodiments, the dose of the Raf inhibitor is about 530 mg/m² per week. In some embodiments, the dose of the Raf inhibitor or a pharmaceutically acceptable salt thereof is about 420 mg/m² per week. In some embodiments, the dose of the Raf inhibitor or a pharmaceutically acceptable salt thereof is about 350 mg/m² per week. In some embodiments, the dose of the Raf inhibitor or a pharmaceutically acceptable salt thereof is about 280 mg/m² per week. In some embodiments, the dose of the Raf inhibitor or a pharmaceutically acceptable salt thereof is about 600 mg/m² to about 700 mg/m² per week. In some embodiments, the dose of the Raf inhibitor or a pharmaceutically acceptable salt thereof is about 500 mg/m² to about 550 mg/m² per week. In some embodiments, the dose of the Raf inhibitor or a pharmaceutically acceptable salt thereof is about 400 mg/m² to about 450 mg/m² per week. In some embodiments, the dose of the Raf inhibitor or a pharmaceutically acceptable salt thereof is about 400 mg/m² to about 500 mg/m² per week. In some embodiments, the dose of the Raf inhibitor or a pharmaceutically acceptable salt thereof is about 200 mg/m² to about 300 mg/m² per week. In some embodiments, the dose of the Raf inhibitor or a pharmaceutically acceptable salt thereof is about 250 mg/m² to about 300 mg/m² per week.

In some embodiments, the Raf inhibitor or a pharmaceutically acceptable salt thereof is administered daily. In some embodiments, the Raf inhibitor or a pharmaceutically acceptable salt thereof is administered every other day. In some embodiments, the Raf inhibitor or a pharmaceutically acceptable salt thereof is administered on a 28-day cycle. In some embodiments, the Raf inhibitor or a pharmaceutically acceptable salt thereof is administered on a 28-day cycle in which the Raf inhibitor or a pharmaceutically acceptable salt thereof is administered on days 1, 3, 5, 8, 10, 12, 15, 17, 19, 22, 24, and 26 of a 28-day cycle. In some embodiments, the Raf inhibitor or a pharmaceutically acceptable salt thereof is administered on a 28-day cycle in which the Raf inhibitor or a pharmaceutically acceptable salt thereof is administered on days 2, 9, 16, and 23 of a 28-day cycle. In some embodiments, the Raf inhibitor or a pharmaceutically acceptable salt thereof is administered for at least 26 cycles. In some embodiments, the Raf inhibitor or a pharmaceutically acceptable salt thereof is administered for at least 5, 6, 7, 8, 9, 10, 11, 12, 13, 14, 15, 16, 17, 18, 19, 20, 21, 22, 23, 24, 25, 26, 27, 28, 29, or 30 cycles. In some embodiments, the Raf inhibitor or a pharmaceutically acceptable salt thereof is administered for at least 20, 21, 22, 23, 24, 25, 26, 27, 28, 29, 30, 31, 32, 33, 34, 35, 36, or 40 cycles. In some embodiments, the Raf inhibitor or a pharmaceutically acceptable salt thereof is administered for at least 24 months, 25 months, 26 months, 27 months, 28 months, 29 months, 30 months, 31 months, 32 months, 33 months, 34 months, 35 months, 36 months, or 48 months.

In some embodiments, Compound A or a pharmaceutically acceptable salt is administered in an amount of up to about 100 mg, 200 mg, 300 mg, 400 mg, 500 mg, about 600 mg, about 700 mg, about 800 mg, about 900 mg, or about 1000 mg of per dose. In some embodiments, Compound A or a pharmaceutically acceptable salt is administered in an amount of up to about 800 mg of Compound A per dose. In some embodiments, Compound A or a pharmaceutically acceptable salt thereof is administered in an amount of about 100 mg to about 800 mg per dose. In some embodiments, Compound A or a pharmaceutically acceptable salt thereof is administered in an amount of about 50 mg to about 1000 mg per dose. In some embodiments, Compound A or a pharmaceutically acceptable salt thereof is administered in an amount of about 50 mg to about 200 mg per dose. In some embodiments, Compound A or a pharmaceutically acceptable salt thereof is administered in an amount of about 500 mg to about 800 mg per dose. In some embodiments, Compound A or a pharmaceutically acceptable salt thereof is administered in an amount of about 100 mg to about 500 mg per dose. In some embodiments, Compound A or a pharmaceutically acceptable salt thereof is administered in an amount of about 400 mg to about 700 mg per dose. In some embodiments, Compound A or a pharmaceutically acceptable salt is administered in an amount of up to about 600 mg per dose. In some embodiments, Compound A or a pharmaceutically acceptable salt is administered in an amount of up to about 400 mg per dose. In some embodiments, Compound A or a pharmaceutically acceptable salt is administered in an amount of up to about 200 mg per dose. In some embodiments, the suitable dosage of Compound A, or a pharmaceutically acceptable salt is from about 100 mg to about 1000 mg per dose. In some embodiments, suitable dosages are about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 220 mg about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, about 500 mg, about 520 mg about 530 mg, about 560 mg, about 580 mg, about 600 mg, about 620 mg, about 640 mg, about 660 mg, about 680 mg, about 700 mg, about 720 mg, about 740 mg, about 760 mg, about 780 mg, about 800 mg per dose, about 820 mg per dose, about 840 mg per dose, about 860 mg per dose, about 880 mg per dose, about 900 mg per dose, about 920 mg per dose, about 940 mg per dose, about 960 mg per dose, about 980 mg per dose, or about 1000 mg per dose. In some embodiments, the Compound A or a pharmaceutically acceptable salt thereof is administered once a month. In some embodiments, the Compound A or a pharmaceutically acceptable salt thereof is administered once a week. In some embodiments, Compound A or a pharmaceutically acceptable salt thereof is administered 2, 3, 4, 5, 6, or 7 times a week.

In some embodiments, Compound A or a pharmaceutically acceptable salt thereof can be administered to a subject in about 100 to about 1000 mg/m² of Compound A per week. In some embodiments, the starting is about 20 mg/m², about 25 mg/m², about 30 mg/m², about 35 mg/m², about 40 mg/m², about 45 mg/m², about 50 mg/m², about 55 mg/m², about 60 mg, about 65 mg/m², about 70 mg/m², about 75 mg/m², about 80 mg/m², about 85 mg/m², about 90 mg/m², about 95 mg/m², about 100 mg/m², about 105 mg/m², about 110 mg/m², about 115 mg/m², about 120 mg/m², about 125 mg/m², about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², about 200 mg/m², about 220 mg/m² about 240 mg/m², about 260 mg/m², about 280 mg/m², about 300 mg/m², about 320 mg/m², about 340 mg/m², about 360 mg/m², about 380 mg/m², about 400 mg/m², about 420 mg/m², about 440 mg/m², about 460 mg/m², about 480 mg/m², about 500 mg/m², about 520 mg/m² about 530 mg/m², about 560 mg/m², about 580 mg/m², about 600 mg/m², about 620 mg/m², about 640 mg/m², about 660 mg/m², about 680 mg/m², about 700 mg/m², about 720 mg/m², about 740 mg/m², about 760 mg/m², about 780 mg/m², about 800 mg/m², about 825 mg/m², about 850 mg/m², about 875 mg/m², about 900 mg/m², about 925 mg/m², about 950 mg/m², about 975 mg/m², or about 1000 mg/m² per week. In some embodiments, the Compound A or a pharmaceutically acceptable salt thereof is administered once a week. In some embodiments, Compound A or a pharmaceutically acceptable salt thereof is administered 2, 3, 4, 5, 6, or 7 times a week. In some embodiments, the dose of Compound A or a pharmaceutically acceptable salt thereof is about 825 mg/m² per week. In some embodiments, the dose of Compound A or a pharmaceutically acceptable salt thereof is about 660 mg/m² per week. In some embodiments, the dose is about 530 mg/m² per week. In some embodiments, the dose of Compound A or a pharmaceutically acceptable salt thereof is about 420 mg/m² per week. In some embodiments, the dose of Compound A or a pharmaceutically acceptable salt thereof is about 350 mg/m² per week. In some embodiments, the dose of Compound A or a pharmaceutically acceptable salt thereof is about 280 mg/m² per week. In some embodiments, the dose of Compound A or a pharmaceutically acceptable salt thereof is about 600 mg/m² to about 700 mg/m² per week. In some embodiments, the dose of Compound A or a pharmaceutically acceptable salt thereof is about 500 mg/m² to about 550 mg/m² per week. In some embodiments, the dose of Compound A or a pharmaceutically acceptable salt thereof is about 400 mg/m² to about 450 mg/m² per week. In some embodiments, the dose of Compound A or a pharmaceutically acceptable salt thereof is about 400 mg/m² to about 500 mg/m² per week. In some embodiments, the dose of Compound A or a pharmaceutically acceptable salt thereof is about 200 mg/m² to about 300 mg/m² per week. In some embodiments, the dose of Compound A or a pharmaceutically acceptable salt thereof is about 250 mg/m² to about 300 mg/m² per week.

In some embodiments, Compound A or a pharmaceutically acceptable salt thereof is administered daily. In some embodiments, Compound A or a pharmaceutically acceptable salt thereof is administered every other day. In some embodiments, Compound A or a pharmaceutically acceptable salt thereof is administered on a 28-day cycle. In some embodiments, Compound A or a pharmaceutically acceptable salt thereof is administered on a 28-day cycle in which Compound A or a pharmaceutically acceptable salt thereof is administered on days 1, 3, 5, 8, 10, 12, 15, 17, 19, 22, 24, and 26 of a 28-day cycle. In some embodiments, Compound A or a pharmaceutically acceptable salt thereof is administered on a 28-day cycle in which Compound A or a pharmaceutically acceptable salt thereof is administered on days 2, 9, 16, and 23 of a 28-day cycle. In some embodiments, Compound A or a pharmaceutically acceptable salt thereof is administered for at least 26 cycles. In some embodiments, Compound A or a pharmaceutically acceptable salt thereof is administered for at least 5, 6, 7, 8, 9, 10, 11, 12, 13, 14, 15, 16, 17, 18, 19, 20, 21, 22, 23, 24, 25, 26, 27, 28, 29, or 30 cycles. In some embodiments, Compound A or a pharmaceutically acceptable salt thereof is administered for at least 20, 21, 22, 23, 24, 25, 26, 27, 28, 29, 30, 31, 32, 33, 34, 35, 36, or 40 cycles. In some embodiments, Compound A or a pharmaceutically acceptable salt thereof is administered for at least 24 months, 25 months, 26 months, 27 months, 28 months, 29 months, 30 months, 31 months, 32 months, 33 months, 34 months, 35 months, 36 months, or 48 months.

### EXAMPLES

The following examples are included merely for purposes of illustration of certain aspects and embodiments of the present disclosure and are not intended to limit the disclosure in any way.

### Example 1: Treatment of Tumors Harboring Gene Fusions Using Raf Kinase Inhibitors

Methods: A male subject, aged 5 years, presented with a 1-week history of fever, cough and respiratory distress. Following worsening shortness of breath and tachypnea, MRI confirmed an 11.2×9.4×11.9 cm dominant right lower hemithorax mass partially encasing the aorta, and likely arising from the posterior mediastinum. Biopsy of the mediastinal mass revealed a diagnosis of spindle cell sarcoma and FISH analysis indicated the presence of a BRAF fusion. The subject was treated with 3 cycles of ifosfamide, doxorubicin, and dexrazoxane. The subject underwent a right thoracotomy with subtotal resection of the right chest mass leaving a 2.1×2.7 cm residual soft tissue mass. Whole exome sequencing of the tumor revealed a novel SNX8: BRAF fusion. The subject was consequently started on the MEK inhibitor trametinib. Following the first month of treatment, there was a decrease in size of residual tumor and hypervascularity resolved. CT was repeated approximately 2 months later and showed no evidence of measurable tumor at the primary site. However, CT was repeated approximately 6 months later and showed a new recurrence of a 4.0×3.7×4.6 cm left posterior mediastinal mass extending circumferentially around the aorta and impressing on the left atrium and pulmonary veins as well as abutting the T7-T8 disc space. Pending molecular test results, which again identified the SNX8: BRAF fusion, the patient started gemcitabine and docetaxel as second-line therapy for recurrent disease. Following 2 cycles, there was no objective response on imaging and symptoms persisted. Compound A was subsequently initiated at 420 mg/m², administered once weekly, in 28-day cycles.

Results: Following 2 cycles of Compound A, symptoms had resolved, and MRI showed no evidence of measurable disease at the site of previously visualized tumor and only a trace amount of non-enhancing soft tissue surrounding the descending thoracic aorta. The subject developed grade 2 rash after the first dose of Compound A, which resolved in 1 day after a dose of diphenhydramine.

Conclusions: Tumors with IFS-like morphology need to undergo comprehensive genomic profiling to identify novel oncogenic fusions. Compound A is potentially an effective treatment in pediatric patients with soft tissue sarcomas harboring BRAF fusions or RAF-1 fusions and warrants further investigation in other BRAF or RAF-1 fusion-driven solid tumors.

### Example 2: In Vivo Efficacy Study: Treatment of HuPrimeR Melanoma Cancer Xenograft Model ME11971 Harboring Gene Fusions in Female NOD/SCID Mice Using Raf Kinase Inhibitors

Methods: The anti-tumor activity of Compound A as a single agent was assessed in female NOD/SCID mice bearing HuPrimeR melanoma cancer xenograft model ME11971. This PDX model harbors the AGK: BRAF fusion as confirmed by RT-PCR.

Mice were implanted SC with ME11971 tumor fragments (2-3 mm in diameter). When mean tumor volume was 151 mm3, mice (n=10/group) were treated with Compound A or vehicle (purified HPLC grade water). Compound A was administered at doses of 12.5, 25, and 50 mg/kg QD×14 days. Inhibition of tumor growth was determined by calculating percent TGI. The T/C value, an indicator of tumor response to treatment which compares the tumor volume of the treatment and control groups was also used to assess anti-tumor activity. Nonparametric tests were run to compare treatment groups with the vehicle control group.

Results: The anti-tumor effects of Compound A are summarized in Table 3. Compound A demonstrated significant anti-tumor activity in the ME11971 model with % TGI of 92.01%, 97.53% and 95.77%, respectively, at doses of 12.5, 25, and 50 mg/kg QD in comparison to vehicle control. Tumor volume was also greatly reduced following treatment with Compound A. Individual body weight loss of over 15% was observed in two animals treated with Compound A, one administered 12.5 mg/kg and one administered 50 mg/kg. The mean body weight loss for these dose groups was less than 10%. There were no mortalities in animals treated with Compound A.

TABLE 3
Anti-tumor Activity of Compound A in
AGK:BRAF Fusion HuPrime ®
Melanoma Xenograft Model ME11971

| Compound | Dosage Regimen | T/C (%) | % TGI | P value |
|---|---|---|---|---|
| Compound A | 12.5 mg/kg QD × 14 | 7.99 | 92.01 | P < .001 |
| | 25 mg/kg QD × 14 | 2.47 | 97.53 | P < .001 |
| | 50 mg/kg QD × 14 | 4.23 | 95.77 | P < .001 |

Abbreviations:
QD = once daily;
T/C = tumor volume in treated animals as a percentage of vehicle control tumor volume;
TGI = tumor growth inhibition

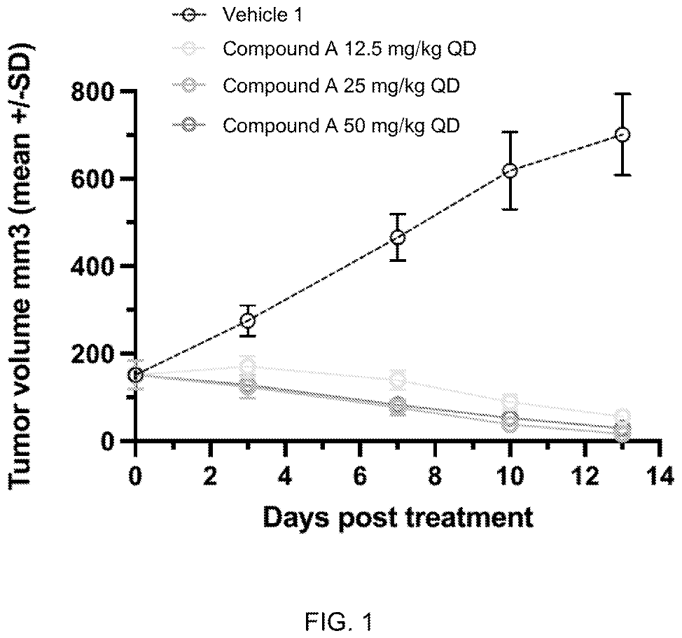

In FIG. 1, plasma concentrations of Compound A were measured 8 hours after the last dose of Compound A administered in tumor-bearing mice. Mean concentrations of Compound A were 5,102±1,064 ng/mL, 12,828±2,283 ng/mL, and 22,350±6,761 ng/mL, respectively, following administration of 12.5, 25, and 50 mg/kg Compound A QD×14 days. Concentrations of Compound A increased dose proportionally.

What is claimed is:

1. A method of treating cancer, the method comprising administering a Raf kinase inhibitor to a subject in need thereof, wherein the subject is identified having a gene fusion selected from AGK: BRAF and SNX8: BRAF.

2. The method of claim 1, wherein the subject is identified having AGK: BRAF gene fusion.

3. The method of claim 1, wherein the subject is identified having SNX8: BRAF gene fusion.

4. The method of claim 1, wherein the Raf kinase inhibitor is (R)-2-(1-(6-amino-5-chloropyrimidine-4-carboxamido)ethyl)-N-(5-chloro-4-(trifluoromethyl) pyridin-2-yl) thiazole-5-carboxamide (Compound A).

5. A method of treating cancer, the method comprising administering a Raf kinase inhibitor to a subject in need thereof, wherein the subject is identified having one or more of the following gene fusions: SNX8: BRAF, STARD3NL: BRAF, BCAS1: BRAF, KHDRBS2: BRAF, CCDC6: BRAF, FAM131B: BRAF, SRGAP: BRAF, CLCN6: BRAF, GNAI1: BRAF, MRKN1: BRAF, GIT2: BRAF, GTF2I: BRAF, FXR1: BRAF, RNF130: BRAF, MACF1: BRAF, TMEM106B: BRAF, PPC1CC: BRAF, CUX1: BRAF, CCD6: BRAF, PPP1CC: BRAF, SEPT7: BRAF, PDE10A: BRAF, EPB41L2: BRAF, OSBP: BRAF, DAAMI: BRAF, TEX41: BRAF, FOXN3: BRAF, TRIPP1: BRAF, TOMIL2: BRAF, 5 BRAF fusions, QK1: RAF1, FYCO: RAF1, ATG7: RAF1, NFIA: RAF1, TMF1: RAF1, GOLGA3: RAF1, SOX6: RAF1, BMPRIA: RAF1, PDZRN3: RAF1, SLMAP: RAF1, MAP4: RAF1, BCL6: RAF1, SEPT17: BRAF, ZNF767: BRAF, CCDC91: BRAF, DYNC1/2: BRAF, ZKSCAN1: BRAF, MZT1: BRAF, RAD18: BRAF, CUX1: RAF1, CUL1: BRAF, SLC12A7: BRAF, TRIM24: BRAF, AGAP3: BRAF, AKAP9: BRAF, TAXIBP1: BRAF, CDC27: BRAF, FKBP15: BRAF, SKAP2: BRAF, TARDBP: BRAF, SEPT3: BRAF, ARMC10: BRAF, PAPSS1: BRAF, FCHSD1: BRAF, ERC1: BRAF, CDK5RAP2: BRAF, TMEM178B: BRAF, BAIAP2L1: BRAF, CEP89: BRAF, CNTNAP2: BRAF, EML4: BRAF, KCTD7: BRAF, LSM14A: BRAF, NFIC: BRAF, NUDCD3: BRAF, PHTF2: BRAF, PLIN3: BRAF, RP2: BRAF, SOX5: BRAF, SOX6: BRAF, TLK2: BRAF, ZKSCAN5: BRAF, KLC1: RAF1, DAAMI: RAF1, ZNF444: RAF1, LRCH3: RAF1, GOLGA4: RAF1, CTDSPL: RAF1, PRKAR2A: RAF1, CTNNA1: RAF1, MKRN1: RAF1, DYNCIH1: RAF1, GPHN: RAF1, KLHL7: BRAF, TANK: BRAF, RBMS3: BRAF, FAM114A2: BRAF, AGGF1: RAF1, EPS15: BRAF, NUP214: BRAF, BTF3L4: BRAF, GHR: BRAF, DOCK4: BRAF, ZC3HAV1: BRAF, MKRN1: BRAF, MYRIP: BRAF, SND1: BRAF, TNS3: BRAF, ATG7: BRAF, NUB1: BRAF, STRN3: BRAF, STK35: BRAF, ETFA: BRAF, SVOPL: BRAF, JHDMID: BRAF, PPFIBP2: BRAF, SCL45A3: BRAF, ESRP1: RAF1, AGTRAP: BRAF, SVIP: BRAF, NRF1: BRAF, RAF1: CCDC176, RAF1: TRAK1, ESYT2: BRAF, PCBP2: BRAF, or SALL2: BRAF.

6. The method of any one of claims 1 to 5, wherein the method comprises identifying the subject having the one or more gene fusions.

7. The method of any one of claims 1 to 6, wherein the method further comprises obtaining a cancer sample from the subject and subjecting the cancer sample to genomic testing prior to the administering of the Raf kinase inhibitor.

8. The method of any one of claims 1 to 7, wherein the RAF kinase inhibitor is selected from: vemurafenib, sorafenib, donafenib, dabrafenib, regorafenib, doramapimod, encorafenib, agerafenib, encorafenib, naporafenib, lifirafenib, belvarafenib, TAK632, CCT3833, RAF265, RAF709, trametinib, cobimetinib, LY3009120, LSN3074753, BGB659, CCT196969, CCT241161, INU152, PLX7904, PLX8394, PLX4720, BI882370, GDC0879, AZ 628, AZ 304, NVP-BHG712, SB590885, MLN2480, L-779450, ZM 336372, GW5074, CEP32496, B-RAF Inhibitor 1, MCP110, BAW2881, RO5126766, and (R)-2-(1-(6-amino-5-chloropyrimidine-4-carboxamido)ethyl)-N-(5-chloro-4-(trifluoromethyl) pyridin-2-yl) thiazole-5-carboxamide (Compound A).

9. The method of any one of claims 1 to 8, wherein the RAF kinase inhibitor is Compound A.

10. A method of treating cancer, the method comprising administering a Raf kinase inhibitor to a subject in need thereof,
wherein the subject has a CRAF gene fusion,
wherein the Raf kinase inhibitor is (R)-2-(1-(6-amino-5-chloropyrimidine-4-carboxamido)ethyl)-N-(5-chloro-4-(trifluoromethyl) pyridin-2-yl) thiazole-5-carboxamide (Compound A) or a pharmaceutically acceptable salt thereof.

11. The method of claim 10, wherein the CRAF gene fusion is SRGAP3: RAF1, QK1: RAF1, FYCO: RAF1, ATG7: RAF1, NFIA: RAF1, TMF1: RAF1, GOLGA3: RAF1, SOX6: RAF1, BMPRIA: RAF1, PDZRN3: RAF1, SLMAP: RAF1, MAP4: RAF1, BCL6: RAF1, CUX1: RAF1, KLC1: RAF1, DAAMI: RAF1, ZNF444: RAF1, LRCH3: RAF1, GOLGA4: RAF1, CTDSPL: RAF1, PRKAR2A: RAF1, CTNNA1: RAF1, MKRN1: RAF1, DYNC1H1: RAF1, AGGF1: RAF1, ESRP1: RAF1, GPHN: RAF1, RAF1-CCDC176, or RAF1-TRAK1.

12. The method of claim 10 or claim 11, wherein the method comprises identifying the subject having the CRAF gene fusion.

13. The method of claim 12, wherein the method further comprises obtaining a cancer sample from the subject and subjecting the cancer sample to genomic testing prior to the administering of Compound A.

14. A method of treating cancer, the method comprising administering a Raf kinase inhibitor to a subject in need thereof,
wherein the subject has one or more of the following gene fusions: KIAA1549: BRAF, AGK: BRAF, STARD3NL: BRAF, BCAS1: BRAF, KHDRBS2: BRAF, CCDC6: BRAF, FAM131B: BRAF, SRGAP: BRAF, CLCN6: BRAF, GNAI1: BRAF, MRKN1: BRAF, GIT2: BRAF, GTF2I: BRAF, FXR1: BRAF, RNF130: BRAF, MACF1: BRAF, TMEM106B: BRAF, PPC1CC: BRAF, CUX1: BRAF, CCD6: BRAF, PPP1CC: BRAF, SEPT7: BRAF, PDE10A: BRAF, EPB41L2: BRAF, OSBP: BRAF, DAAMI: BRAF, TEX41: BRAF, FOXN3: BRAF, TRIPP1: BRAF, TOMIL2: BRAF, TMEM106B: BRAF, SRGAP3: RAF1, QK1: RAF1, FYCO: RAF1, ATG7: RAF1, NFIA-RAF1, TMF1: RAF1, GOLGA3: RAF1, SOX6: RAF1, BMPRIA: RAF1, PDZRN3: RAF1, SLMAP: RAF1, MAP4: RAF1, BCL6-RAF1, SEPT17: BRAF, ZNF767: BRAF, CCDC91: BRAF, DYNC1/2: BRAF, ZKSCAN1: BRAF, GTF2I: BRAF, MZT1: BRAF, RAD18: BRAF, CUX1: BRAF, CUX1-RAF1, CUL1: BRAF, SLC12A7: BRAF, TRIM24: BRAF, AGAP3: BRAF, AKAP9: BRAF, TAXIBP1: BRAF, CDC27: BRAF, FKBP15: BRAF, SKAP2: BRAF, TARDBP: BRAF, SEPT3: BRAF, ARMC10: BRAF, PAPSS1: BRAF, FCHSD1: BRAF, ERC1: BRAF, CDK5RAP2: BRAF, TMEM178B: BRAF, BAIAP2L1: BRAF, CEP89: BRAF, CNTNAP2: BRAF, EML4: BRAF, KCTD7: BRAF, LSM14A: BRAF, NFIC: BRAF, NUDCD3: BRAF, PHTF2: BRAF, PLIN3: BRAF, RP2: BRAF, SOX5: BRAF, SOX6: BRAF, TLK2: BRAF, ZKSCAN5: BRAF, KLC1-RAF1, DAAMI-RAF1, ZNF444-RAF1, LRCH3-RAF1, GOLGA4-RAF1, CTDSPL-RAF1, PRKAR2A-RAF1, CTNNA1-RAF1, MKRN1-RAF1, DYNCIH1-RAF1, GPHN-RAF1, KLHL7: BRAF, TANK: BRAF, RBMS3: BRAF, FAM114A2: BRAF, AGGF1-RAF1, EPS15: BRAF, NUP214: BRAF, BTF3L4: BRAF, GHR: BRAF, DOCK4: BRAF, ZC3HAV1: BRAF, MKRN1: BRAF, MYRIP: BRAF, SND1: BRAF, TNS3: BRAF, ATG7: BRAF, NUB1: BRAF, STRN3: BRAF, STK35: BRAF, ETFA: BRAF, SVOPL: BRAF, JHDMID: BRAF, PPFIBP2: BRAF, SCL45A3: BRAF, ESRP1-RAF1, AGTRAP: BRAF, SVIP: BRAF, NRF1: BRAF, RAF1-CCDC176, RAF1-TRAK1, ESYT2: BRAF, PCBP2: BRAF, and SALL2: BRAF,
wherein the Raf kinase inhibitor is (R)-2-(1-(6-amino-5-chloropyrimidine-4-carboxamido)ethyl)-N-(5-chloro-4-(trifluoromethyl) pyridin-2-yl) thiazole-5-carboxamide (Compound A) or a pharmaceutically acceptable salt thereof, and
wherein the subject is at least 18 years of age.

15. A method of treating a solid tumor in an adult subject, the method comprising administering a Raf kinase inhibitor to a subject in need thereof,
wherein the subject has a CRAF gene fusion, a BRAF gene fusion, or both,
wherein the Raf kinase inhibitor is (R)-2-(1-(6-amino-5-chloropyrimidine-4-carboxamido)ethyl)-N-(5-chloro-4-(trifluoromethyl) pyridin-2-yl) thiazole-5-carboxamide (Compound A) or a pharmaceutically acceptable salt thereof,
wherein the subject is at least 18 years of age.

16. The method of claim 15, wherein the subject has a gene fusion that is KIAA1549: BRAF or SRGAP3: RAF1.

17. A method of treating cancer, the method comprising administering a Raf kinase inhibitor to a subject in need thereof,
wherein the subject has one or more of the following gene fusions: AGK: BRAF, STARD3NL: BRAF, BCAS1: BRAF, KHDRBS2: BRAF, CCDC6: BRAF, FAM131B: BRAF, SRGAP: BRAF, CLCN6: BRAF, GNAI1: BRAF, MRKN1: BRAF, GIT2: BRAF, GTF2I: BRAF, FXR1: BRAF, RNF130: BRAF, MACF1: BRAF, TMEM106B: BRAF, PPC1CC: BRAF, CUX1: BRAF, CCD6: BRAF, PPP1CC: BRAF, SEPT7: BRAF, PDE10A: BRAF, EPB41L2: BRAF, OSBP: BRAF, DAAMI: BRAF, TEX41: BRAF, FOXN3: BRAF, TRIPP1: BRAF, TOMIL2: BRAF, TMEM106B: BRAF, QK1: RAF1, FYCO: RAF1, ATG7: RAF1, NFIA-RAF1, TMF1: RAF1, GOLGA3: RAF1, SOX6: RAF1, BMPRIA: RAF1, PDZRN3: RAF1, SLMAP: RAF1, MAP4: RAF1, BCL6-RAF1, SEPT17: BRAF, ZNF767: BRAF, CCDC91: BRAF, DYNC1/2: BRAF, ZKSCAN1: BRAF, GTF2I: BRAF, MZT1: BRAF, RAD18: BRAF, CUX1: BRAF, CUX1-RAF1, CUL1: BRAF, SLC12A7: BRAF, TRIM24: BRAF, AGAP3: BRAF, AKAP9: BRAF, TAXIBP1: BRAF, CDC27: BRAF, FKBP15: BRAF, SKAP2: BRAF, TARDBP: BRAF, SEPT3: BRAF, ARMC10: BRAF, PAPSS1: BRAF, FCHSD1: BRAF, ERC1: BRAF, CDK5RAP2: BRAF, TMEM178B: BRAF, BAIAP2L1: BRAF, CEP89: BRAF, CNTNAP2: BRAF, EML4: BRAF, KCTD7: BRAF, LSM14A: BRAF, NFIC: BRAF, NUDCD3: BRAF, PHTF2: BRAF, PLIN3: BRAF, RP2: BRAF, SOX5: BRAF, SOX6: BRAF, TLK2: BRAF, ZKSCAN5: BRAF, KLC1-RAF1, DAAMI-RAF1, ZNF444-RAF1, LRCH3-RAF1, GOLGA4-RAF1, CTDSPL-RAF1, PRKAR2A-RAF1, CTNNA1-RAF1, MKRN1-RAF1, DYNCIH1-RAF1, GPHN-RAF1, KLHL7: BRAF, TANK: BRAF, RBMS3: BRAF, FAM114A2: BRAF, AGGF1-RAF1, EPS15: BRAF, NUP214: BRAF, BTF3L4: BRAF, GHR: BRAF, DOCK4: BRAF, ZC3HAV1: BRAF, MKRN1: BRAF, MYRIP: BRAF, SND1: BRAF, TNS3: BRAF, ATG7: BRAF, NUB1: BRAF, STRN3: BRAF, STK35: BRAF, ETFA: BRAF, SVOPL: BRAF, JHDMID: BRAF, PPFIBP2: BRAF, SCL45A3: BRAF, ESRP1-RAF1, AGTRAP: BRAF, SVIP: BRAF, NRF1: BRAF, RAF1-CCDC176, RAF1-TRAK1, ESYT2: BRAF, PCBP2: BRAF, or SALL2: BRAF, and
wherein the Raf kinase inhibitor is (R)-2-(1-(6-amino-5-chloropyrimidine-4-carboxamido)ethyl)-N-(5-chloro-4-(trifluoromethyl) pyridin-2-yl) thiazole-5-carboxamide (Compound A) or a pharmaceutically acceptable salt thereof.

18. The method of claim 17, wherein the gene fusion is a BRAF fusion.

19. The method of claim 17 or claim 18, wherein the BRAF fusion is selected from:
AGK: BRAF, STARD3NL: BRAF, BCAS1: BRAF, KHDRBS2: BRAF, CCDC6: BRAF, FAM131B: BRAF, SRGAP: BRAF, CLCN6: BRAF, GNAI1: BRAF, MRKN1: BRAF, GIT2: BRAF, GTF2I: BRAF, FXR1: BRAF, RNF130: BRAF, MACF1: BRAF, TMEM106B: BRAF, PPC1CC: BRAF, CUX1: BRAF, CCD6: BRAF, PPP1CC: BRAF, SEPT7: BRAF, PDE10A: BRAF, EPB41L2: BRAF, OSBP: BRAF, DAAMI: BRAF, TEX41: BRAF, FOXN3: BRAF, TRIPP1: BRAF, TOMIL2: BRAF, 5 BRAF fusions, TMEM106B: BRAF, SEPT17: BRAF, ZNF767: BRAF, CCDC91: BRAF, DYNC1/2: BRAF, ZKSCAN1: BRAF, GTF2I: BRAF, MZT1: BRAF, RAD18: BRAF, CUX1: BRAF, CUL1: BRAF, SLC12A7: BRAF, TRIM24: BRAF, AGAP3: BRAF, AKAP9: BRAF, TAXIBP1: BRAF, CDC27: BRAF, FKBP15: BRAF, SKAP2: BRAF, TARDBP: BRAF, SEPT3: BRAF, ARMC10: BRAF, PAPSS1: BRAF, FCHSD1: BRAF, ERC1: BRAF, CDK5RAP2: BRAF, TMEM178B: BRAF, BAIAP2L1: BRAF, CEP89: BRAF, CNTNAP2: BRAF, EML4: BRAF, KCTD7: BRAF, LSM14A: BRAF, NFIC: BRAF, NUDCD3: BRAF, PHTF2: BRAF, PLIN3: BRAF, RP2: BRAF, SOX5: BRAF, SOX6: BRAF, TLK2: BRAF, ZKSCAN5: BRAF, KLHL7: BRAF, TANK: BRAF, RBMS3: BRAF, FAM114A2: BRAF, EPS15: BRAF, NUP214: BRAF, BTF3L4: BRAF, GHR: BRAF, DOCK4: BRAF, ZC3HAV1: BRAF, MKRN1: BRAF, MYRIP: BRAF, SND1: BRAF, TNS3: BRAF, ATG7: BRAF, NUB1: BRAF, STRN3: BRAF, STK35: BRAF, ETFA: BRAF, SVOPL: BRAF, JHDMID: BRAF, PPFIBP2: BRAF, SCL45A3: BRAF, AGTRAP: BRAF, SVIP: BRAF, NRF1: BRAF, ESYT2: BRAF, PCBP2: BRAF, and SALL2: BRAF.

20. The method of claim 17, wherein the gene fusion is a RAF1 fusion.

21. The method of claim 17 or claim 20, wherein the RAF1 fusion is selected from:
SRGAP3: RAF1, QK1: RAF1, FYCO: RAF1, ATG7: RAF1, NFIA: RAF1, TMF1: RAF1, GOLGA3: RAF1, SOX6: RAF1, BMPRIA: RAF1, PDZRN3: RAF1, SLMAP: RAF1, MAP4: RAF1, BCL6: RAF1, CUX1: RAF1, KLC1: RAF1, DAAMI: RAF1, ZNF444: RAF1, LRCH3: RAF1, GOLGA4: RAF1, CTDSPL: RAF1, PRKAR2A: RAF1, CTNNA1: RAF1, MKRN1: RAF1, DYNCIH1: RAF1, AGGF1: RAF1, ESRP1: RAF1, GPHN: RAF1, RAF1-CCDC176, and RAF1-TRAK1.

22. A method of treating a solid tumor in an adult subject, the method comprising administering a Raf kinase inhibitor to a subject in need thereof,
wherein the subject has a wild-type CRAF gene fusion, a wild-type BRAF gene fusion, or both,
and wherein the Raf kinase inhibitor is (R)-2-(1-(6-amino-5-chloropyrimidine-4-carboxamido)ethyl)-N-(5-chloro-4-(trifluoromethyl) pyridin-2-yl) thiazole-5-carboxamide (Compound A) or a pharmaceutically acceptable salt thereof.

23. The method of any one of claims 1 to 21, wherein the cancer is a solid tumor.

24. The method of claim 22 or 23, wherein the solid tumor is an extracranial tumor.

25. The method of claim 24, wherein the solid tumor is a soft tissue sarcoma.

26. The method of claim 24, wherein the solid tumor is a bone sarcoma.

27. The method of claim 26, wherein the bone sarcoma is selected from osteosarcoma, chonrosarcoma, spindle cell sarcoma, hemangioendothelioma, angiosarcoma, fibrosarcoma, chordoma, adamantinoma, liposarcoma, leiomyosarcoma, malignant peripheral nerve sheath tumor, rhabdomyosarcoma, synovial sarcoma, and malignant solitary fibrous tumor.

28. The method of claim 26 or claim 27, wherein the bone sarcoma is spindle cell sarcoma.

29. The method of any one of claims 1 to 28, wherein the cancer is an advanced solid tumor.

30. The method of any one of claims 1 to 29, wherein the cancer is a recurrent cancer.

31. The method of any one of claims 8 to 30, wherein the subject has received at least one prior therapy before the administration of the (R)-2-(1-(6-amino-5-chloropyrimidine-4-carboxamido)ethyl)-N-(5-chloro-4-(trifluoromethyl) pyridin-2-yl) thiazole-5-carboxamide (Compound A) or a pharmaceutically acceptable salt thereof.

32. The method of claim 31, wherein the prior therapy is chemotherapy therapy, hormone therapy, immunotherapy, or radiation therapy.

33. The method of any one of claims 14 to 32, wherein the method further comprises obtaining a cancer sample from the subject and subjecting the cancer sample to genomic profiling prior to the administering of Compound A.

34. The method of claim 33, wherein the genomic profiling is whole exome sequencing.

35. The method of any one of claims 1 to 34, wherein the method further comprises the administration of a dermatological agent.

36. The method of claim 35, wherein the dermatological agent is diphenhydramine.

37. The method of any one of claims 8 to 36, wherein the administrating occurs in a plurality of treatment stages which alternate between an administration stage and a non-administration stage.

38. The method of claim 37, wherein the administration stage has a duration of 28 days.

39. The method of claim 37 or claim 38, wherein the administration stage comprises the administration Compound A or a pharmaceutically acceptable salt thereof once weekly.

40. The method of any one of claims of 37 to 39, wherein Compound A or a pharmaceutically acceptable salt thereof is administered at 420 mg/m².

41. The method of any one of claims 8 to 39, wherein the administering of Compound A or a pharmaceutically acceptable salt thereof, comprises an initial dose of the Compound A or a pharmaceutically acceptable salt thereof equivalent to about 280 mg/m² to about 600 mg/m² of Compound A per week.

42. The method of any one of claims 8 to 39, wherein Compound A or a pharmaceutically acceptable salt thereof, is administered in an amount of up to about 600 mg per dose.

43. The method of claim 42, wherein Compound A or a pharmaceutically acceptable salt thereof, is administered in an amount of up to about 200 mg per dose.

44. The method of claim 42 or claim 43, wherein Compound A or a pharmaceutically acceptable salt thereof, is administered on days 1, 3, 5, 8, 10, 12, 15, 17, 19, 22, 24, and 26 of a 28-day cycle.
